(19) United States
(12) Patent Application Publication
(10) Pub. No.: US 2020/0277559 A1
(43) Pub. Date: Sep. 3, 2020

(54) Expressors and Expressor Systems for Separating Components of a Biological Suspension and Methods of Use

(71) Applicant: Thermo Electron LED GmbH, Langenselbold (DE)

(72) Inventors: Jason D. Brown, Logan, UT (US); Romana Hinz, Windhausen (DE)

(21) Appl. No.: 16/557,736

(22) Filed: Aug. 30, 2019

Related U.S. Application Data
(63) Continuation-in-part of application No. 16/289,296, filed on Feb. 28, 2019.

(51) Int. Cl. C12M 1/26; B04B 5/04; C12M 1/00

(52) U.S. Cl. CPC C12M 33/10; B04B 2005/0435; C12M 23/14; B04B 5/0407

(57) ABSTRACT

An expressor includes a base and a first platen extending from the base. A second platen is movably mounted to the base so that the second platen can move between a collapsed position where the second platen is moved toward the first platen and a retracted position where the second platen is moved away from the first platen. At least a portion of the second platen is spaced apart from the first platen by a gap spacing when the second platen is in the collapsed position. Either the first platen or the second platen is releasably attached to the base so that a width of the gap spacing when the second platen is in the collapsed position can be selectively adjusted.

### CROSS-REFERENCE TO RELATED APPLICATIONS

This application is a continuation-in-part of U.S. application Ser. No. 16/289,296, filed Feb. 28, 2019, which is incorporated herein by specific reference.

### BACKGROUND

### 1. The Field of the Disclosure

The present disclosure relates to expressors, expressor systems, and related methods for driving a liquid supernatant out of a collapsible bag that also houses a pellet comprised of cells or microorganisms.

### 2. The Relevant Technology

Bioreactors and fermenters are used to grow a variety of different types of biological suspensions. Such suspensions are broadly defined as comprising cells or microorganisms and a liquid medium in which they are suspended. Once a suspension has been sufficiently grown, it is common to separate the biological suspension into components and then harvest the separate components for subsequent analysis or use. Centrifugation is a technique often employed during isolation or analysis of various cells, organelles, and biopolymers, including proteins, nucleic acids, lipids, and carbohydrates dissolved or dispersed in biological suspension.

In one approach to centrifugation, quantities of a suspension are dispensed from a bioreactor or fermenter into an open-top bottle. The bottle is then closed by manually applying a lid and then spun using a centrifuge rotor. The centrifugal force created by spinning of the rotor causes the solids within the suspension to sediment out of the solution to form a generally solid pellet towards the bottom of the bottle. A supernatant, which is a liquid that is less dense than the pellet, collects within the bottle above the pellet. The supernatant is then decanted from the bottle by removing the lid and then pouring and/or pumping out the supernatant. The pellet can then be separately removed from the bottle.

Although the above process is effective, it has a number of shortcomings. For example, the bottles that are used as open-top containers. Thus, both the suspension and the interior of the bottles are openly exposed to the surrounding environment as the suspension is initially dispensed into the bottles. In turn, the separated components are again openly exposed to the surrounding environment as the separated components are removed from the bottles. This open exposure to the environment increases the probability of the suspension and/or the separated components becoming contaminated. Subsequent purification steps can thus be required to remove any contaminates from one or both of the separated components. Conventional methods and systems thus have a high probability of contamination and can require added labor, time, and cost to run purification steps.

In addition to the above, it can be difficult in conventional systems to effectively separate the supernatant from the pellet. That is, it is typically desirable to maximize the quantity of cells or microorganism within the pellet and to minimize the quantity of cells or microorganism within the supernatant. However, in some applications the pellet can be easily disturbed causing the solids thereof to resuspend into the supernatant. As such, it can be a slow and labor intensive process to carefully decant the supernatant from the bottles without disturbing the pellet. It is often necessary to sacrifice some supernatant to avoid disturbing the pellet.

In one attempt to solve some of the above problems, a removable, open-top liner has been placed within a centrifuge container. The liner bounds the compartment of the container and receives the biological suspension. Following use, the liner is discarded and a new liner can be inserted within the centrifuge container without the need for cleaning or sterilization of the container. The liner, like the above discussed bottle, is open and exposed to the surrounding environment during dispensing of the biological suspension therein. As such, there remains an increased risk of the suspension and components being contaminated and the need for purification steps. Furthermore, the liner does not solve the difficulty of separating the supernatant from the pellet. Other shortcomings also exist.

Accordingly, what is needed in the art are improved system and methods that solve all or some of the above and other existing shortcomings.

### SUMMARY OF THE DISCLOSURE

In a first independent aspect of the present disclosure, an expressor includes:
a base;
a first platen having an inside face extending between an upper end and an opposing lower end, the lower end extending from the base;
a second platen having an inside face extending between an upper end and an opposing lower end, the second platen being movably mounted to the base so that the second platen can move between a collapsed position where the second platen is moved toward the first platen and a retracted position where the second platen is moved away from the first platen, at least a portion of the second platen being spaced apart from the first platen by a gap spacing when the second platen is in the collapsed position,
wherein either the first platen or the second platen is releasably attached to the base so that a width of the gap spacing when the second platen is in the collapsed position can be selectively adjusted.

In one embodiment, the lower end of the second platen is pivotably connected to the base so that the second platen can pivot between the collapsed position and the retracted position.

In another embodiment, the second platen moves laterally without pivoting as it moves between the collapsed position and the retracted position.

In another embodiment, either the first platen or the second platen is releasably attached to the base so that the width of the gap spacing can be selectively adjusted without pivoting of the first platen or the second platen.

In another embodiment, the first platen or the second platen can be moved laterally relative to the other platen to selectively adjust the width of the gap spacing.

In another embodiment, the first platen is releasably attached to the base so that the width of the gap spacing can be selectively adjusted.

In another embodiment, a releasable fastener releasably attaches the first platen to the base.

In another embodiment, a first opening passes through a portion of the first platen, the releasable fastener passing through the first opening.

In another embodiment, the inside face of the first platen is disposed in parallel alignment with the inside face of the second platen when the second platen is in the collapsed position.

In another embodiment, the first platen or the second platen is movably attached to the base so that the width of the gap spacing can be selectively adjusted by at least 0.25 cm, 0.5 cm, 1 cm, or 2 cm.

In another embodiment, the second platen is pivotably connected to the base by a hinge.

In another embodiment, means are provided for moving the second platen toward the first platen.

In another embodiment, the means for moving resiliently urges the second platen toward the first platen.

In another embodiment, the means for moving includes a spring, pneumatic piston or hydraulic piston.

In another embodiment, the first platen inwardly tapers at the lower end.

In another embodiment, the entire first platen is spaced apart from the second platen when the second platen is in the collapsed position.

In another embodiment, an expressor system includes:
the expressor; and
a bag assembly including:
   a collapsible bag bounding a compartment that is adapted to hold a fluid, the bag being disposed between the first platen and the second platen; and
   a tube projecting from the collapsible bag.

In another embodiment, a pellet comprised of cells or microorganisms is disposed within the compartment of the bag and a liquid supernatant is disposed within the compartment of the bag.

In another embodiment, the pellet includes cells that are free red blood cells and white blood cells.

In another embodiment, the liquid supernatant is free of plasma.

In another embodiment, an optical sensor and a pinch clamp are each disposed on or overlay the tube projecting from the collapsible bag.

In another embodiment, a method is provided for using the expressor for removing a supernatent from a compartment of a collapsible bag that houses the supernatant and a pellet comprised of cells or microorganisms, the method includes:
moving the first platen or the second platen of the expressor relative to the base so as to adjust the width of the gap spacing between the first platen and the second platen based upon an amount of pellet within the compartment of the bag;
positioning the collapsible bag between the first platen and the second platen; and
moving the second platen toward the first platen so as to compress the bag between the first platen and the second platen and drive at least a portion of the supernatant out of the bag through a tube coupled with the bag.

In a second independent aspect of the present disclosure, an expressor includes:
a base;
a first platen having an inside face and an opposing outside face extending between an upper end and an opposing lower end, the upper end having a perimeter edge with a first notch recessed into the perimeter edge so that the first notch passes between the inside face and the outside face, the lower end being connected to the base;
a second platen having an inside face extending between an upper end and an opposing lower end, the lower end of the second platen being movably mounted to the base so that the second platen can move between a collapsed position where the second platen is moved toward the first platen and a retracted position where the second platen is moved away from the first platen.

In another embodiment, the second platen is spaced apart from the first platen by a gap spacing when the second platen is in the collapsed position.

In another embodiment, the second platen is pivotably connected to the base so that the second platen can pivot between the collapsed position and the retracted position.

In another embodiment, the second platen is pivotably connected to the base by a hinge.

In another embodiment, the first notch has a width in a range between 0.5 cm and 3 cm.

In another embodiment, a second notch is recessed into the perimeter edge at the upper end of the first platen so that the second notch passes between the inside face and the outside face, the second notch being spaced apart from the first notch.

In another embodiment, means are provided for moving the second platen between the collapsed position and the retracted position.

In another embodiment, the first platen inwardly tapers at the lower end.

In another embodiment, an expressor system includes:
the expressor; and
a bag assembly including:
   a collapsible bag bounding a compartment that is adapted to hold a fluid, the bag having a front face and an opposing back face, the bag being disposed between the first platen and the second platen;
   a first port being disposed on the front face of the bag and communicating with the compartment, the first port being received within the first notch of the first platen;
   a tube projecting from the first port.

In another embodiment, a pellet comprised of cells or microorganisms is disposed within the compartment of the bag and a liquid supernatant is disposed within the compartment of the bag.

In another embodiment, the pellet includes cells that are free red blood cells and white blood cells.

In another embodiment, the liquid supernatant is free of plasma.

In another embodiment, the expressor system further includes:
the first platen further comprising a second notch recessed into the perimeter edge at the upper end of the first platen so that the second notch passes between the inside face and the outside face, the second notch being spaced apart from the first notch; and
the bag assembly further comprising a second port being disposed on the front face of the bag and communicating with the compartment, the second port being received within the second notch of the first platen.

In another embodiment, a method is provided for using the expressor for removing a supernatant from a compartment of a collapsible bag that houses the supernatant and a pellet comprised of cells or microorganisms, the method includes:
positioning the collapsible bag between the first platen and the second platen of the expressor, a first port being disposed on the front face of the bag and communicating with the compartment, the first port being received within the first notch of the first platen; and
moving the second platen toward the first platen so as to compress the bag between the first platen and the second platen and drive at least a portion of the supernatant out of the bag through a tube coupled with the bag.

In a third independent aspect of the present disclosure, an expressor includes:
a base;
a first platen having an inside face extending between an upper end and an opposing lower end, the lower end extending from the base;
a first arm and a spaced apart second arm movably mounted to the base;
a second platen having an inside face extending between an upper end and an opposing lower end, the second platen being pivotably connected to the first arm and the second arm so that the second platen can pivot toward and away from the first platen.

In another embodiment, the first arm and the second arm each have a first end and an opposing second end, the first end of each arm being pivotably connected to the base.

In another embodiment, the second platen is pivotably connected to the second end of each arm.

In another embodiment, the first arm and the second arm can each adjust in length.

In another embodiment, a spring resiliently urges the second platen toward the first platen.

In another embodiment, an expressor system includes:
the expressor; and
a bag assembly including a collapsible bag bounding a compartment that is adapted to hold a liquid, the bag being disposed between the first platen and the second platen, the second platen being elevated by the first arm and the second arm so as to be spaced apart from the base.

In another embodiment, a pellet comprised of cells or microorganisms is disposed within the compart of the bag and a liquid supernatant is disposed within the compartment of the bag.

In another embodiment, the pellet includes cells that are free red blood cells or white blood cells.

In another embodiment, the liquid supernatant is free of plasma.

In another embodiment, the lower end of the second platen presses against the bag at a location above the pellet.

In another embodiment, a clamp is clamped across the bag at a location above the pellet.

In another embodiment, a method is provided for using the expressor for removing a supernatant from a compartment of a collapsible bag that houses the supernatant and a pellet comprised of cells or microorganisms, the method includes:
positioning the collapsible bag between the first platen and the second platen of the expressor;
moving the first arm and the second arm so as to elevate the second platen;
urging a hinge connected to second platen against the bag at a location above the pellet; and
moving the second platen toward the first platen so as to compress the bag between the first platen and the second platen and drive at least a portion of the supernatant out of the bag through a tube coupled with the bag.

In a fourth independent aspect of the present disclosure, an expressor system includes:
an expressor having:
   a base;
   a first platen having an inside face extending between an upper end and an opposing lower end, the lower end extending from the base; and
   a second platen having an inside face extending between an upper end and an opposing lower end, the second platen being movably mounted to the base so that the second platen can move between a collapsed position where the second platen is moved toward the first platen and a retracted position where the second platen is moved away from the first platen, at least a portion of the second platen being spaced apart from the first platen by a gap spacing when the second platen is in the collapsed position,
a collapsible bag bounding a compartment that is adapted to hold a fluid, the bag being disposed between the first platen and the second platen; and
a pellet and a liquid supernatant being disposed within the compartment of the bag, the pellet being comprised of cells or microorganisms and being free of red blood cells or white blood cells.

In one embodiment, the liquid supernatant is free of plasma.

In another embodiment, a method is provided for using the expressor system, the method including:
moving the second platen toward the first platen so as to compress the bag between the first platen and the second platen and drive at least a portion of the supernatant out of the bag through a tube coupled with the bag; and
removing the bag from between the first platen and the second platen.

Each of the above independent aspects of the disclosure may include any of the features, options and possibilities set out in this document, including those under the other independent aspects, and may also include any combination of any of the features, options and possibilities set out in this document.

### BRIEF DESCRIPTION OF THE DRAWINGS

Various embodiments of the present disclosure will now be discussed with reference to the appended drawings. It is appreciated that these drawings depict only typical embodiments of the disclosure and are therefore not to be considered limiting of its scope.

FIG. 1 is an elevated front view of a reactor that is fluid coupled with a bag assembly;

FIG. 2 is an elevated front view of the bag assembly shown in FIG. 1;

FIG. 3 is an exploded view of the bag assembly shown in FIG. 2;

FIG. 4 is an elevated front view of an alternative embodiment of the bag assembly shown in FIG. 2;

FIG. 5 is an exploded view of the bag assembly shown in FIG. 4;

FIG. 6 is an elevated front view of the bag assembly shown in FIG. 1 including an inlet line and an outlet line;

FIG. 7 is a perspective view of one embodiment of a centrifuge (floor standing model) that can be used in the present disclosure;

FIG. 8 is an elevated front view of the bag assembly shown in FIG. 6 after being removed from the centrifuge;

FIG. 9 is an elevated front view of the bag assembly shown in FIG. 8 fluid coupled with a container in which the supernatant is to be disposed;

FIG. 10 is a front perspective view of an expressor in a retracted position;

FIG. 11 is a rear perspective view of the expressor shown in FIG. 10;

FIG. 12 is an enlarged view of the section identified in FIG. 11;

FIG. 13 is a rear perspective view of the expressor shown in FIG. 10 in a collapsed position;

FIG. 14 a rear perspective view of the expressor shown in FIG. 13 with the first platen thereof moved to a second position;

FIG. 15 is an enlarged perspective view of an alternative embodiment of an expressor having releasable cams;

FIG. 16 is a perspective view of the expressor shown in FIG. 10 compressing the bag assembly shown in FIG. 4 that is coupled to a container;

FIG. 17 is a perspective view of the assembly shown in FIG. 16 being used with an optical sensor, an electronic pinch clamp and a processor;

FIG. 18 is a perspective view of an alternative embodiment of the expressor shown in FIG. 10 being operated by a piston;

FIG. 19 is an elevated side view of an alternative embodiment of an expressor wherein the second platen moves laterally;

FIG. 20 is a front perspective view of an alternative embodiment of an expressor supporting the bag assembly of FIG. 4;

FIG. 21 is a front perspective view of the expressor of FIG. 20 compressing the bag assembly;

FIG. 22 is an elevated front view of the bag assembly with container shown in FIG. 9 and having a clamp mounted on the bag assembly; and

FIG. 23 is a front perspective view of the expressor shown in FIG. 20 having the clamp of FIG. 22 mounted on the bag assembly.

### DETAILED DESCRIPTION OF THE PREFERRED EMBODIMENTS

Before describing the present disclosure in detail, it is to be understood that this disclosure is not limited to particularly exemplified apparatus, systems, methods, or process parameters that may, of course, vary. It is also to be understood that the terminology used herein is only for the purpose of describing particular embodiments of the present disclosure and is not intended to limit the scope of the disclosure in any manner.

All publications, patents, and patent applications cited herein, whether supra or infra, are hereby incorporated by reference in their entirety to the same extent as if each individual publication, patent, or patent application was specifically and individually indicated to be incorporated by reference.

The term “comprising” which is synonymous with “including,” “containing,” or “characterized by,” is inclusive or open-ended and does not exclude additional, unrecited elements or method steps.

It will be noted that, as used in this specification and the appended claims, the singular forms “a,” “an” and “the” include plural referents unless the content clearly dictates otherwise. Thus, for example, reference to a “port” includes one, two, or more ports.

As used in the specification and appended claims, directional terms, such as “top,” “bottom,” “left,” “right,” “up,” “down,” “upper,” “lower,” “proximal,” “distal” and the like are used herein solely to indicate relative directions and are not otherwise intended to limit the scope of the disclosure or claims.

Where possible, like numbering of elements have been used in various figures. Furthermore, multiple instances of an element and or sub-elements of a parent element may each include separate letters appended to the element number. For example, two instances of a particular element “10” may be labeled as “10A” and “10B”. In that case, the element label may be used without an appended letter (e.g., “10”) to generally refer to all instances of the element or any one of the elements. Element labels including an appended letter (e.g., “10A”) can be used to refer to a specific instance of the element or to distinguish or draw attention to multiple uses of the element. Furthermore, an element label with an appended letter can be used to designate an alternative design, structure, function, implementation, and/or embodiment of an element or feature without an appended letter. Likewise, an element label with an appended letter can be used to indicate a sub-element of a parent element. For instance, an element “12” can comprise sub-elements “12A” and “12B.”

Various aspects of the present devices and systems may be illustrated by describing components that are coupled, attached, and/or joined together. As used herein, the terms “coupled”, “attached”, and/or “joined” are used to indicate either a direct connection between two components or, where appropriate, an indirect connection to one another through intervening or intermediate components. In contrast, when a component is referred to as being “directly coupled”, “directly attached”, and/or “directly joined” to another component, there are no intervening elements present. Furthermore, as used herein, the terms “connection,” “connected,” and the like do not necessarily imply direct contact between the two or more elements.

Various aspects of the present devices, systems, and methods may be illustrated with reference to one or more exemplary embodiments. As used herein, the term “embodiment” means “serving as an example, instance, or illustration,” and should not necessarily be construed as preferred or advantageous over other embodiments disclosed herein.

Unless defined otherwise, all technical and scientific terms used herein have the same meaning as commonly understood by one of ordinary skill in the art to which the present disclosure pertains. Although a number of methods and materials similar or equivalent to those described herein can be used in the practice of the present disclosure, the preferred materials and methods are described herein.

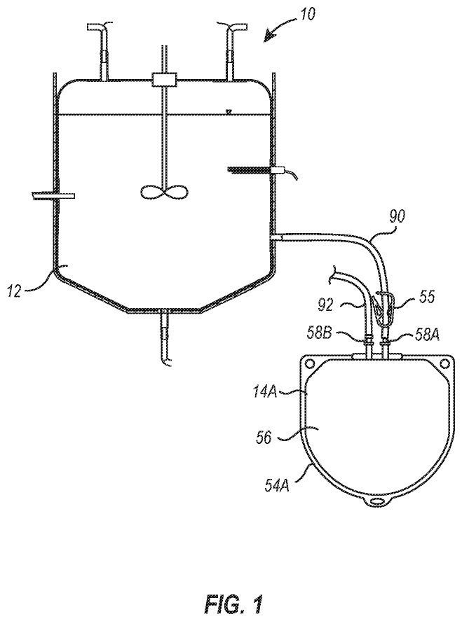

In general, present disclosure relates to expressors, expressor systems, and methods for driving a liquid supernatant out of a collapsible bag that also houses a pellet comprised of cells or microorganisms. The supernatant and pellet are typically derived from a biological suspension that is grown in a reactor. For example, with reference to FIG. 1, a reactor 10 is provided for growing a biological suspension 12. Reactor 10 can comprise a bioreactor, fermenter, or any other device designed for growing or producing biological suspensions. The term “bioreactor” as used herein is broadly intended to cover multi-plate growth chambers such as the Cell Factory multi-plate growth chamber produced by Thermo Fisher Scientific. It is also appreciated that reactor 10 can comprise any conventional type of bioreactor or fermenter such as a stirred-tank reactor, rocker type reactor, paddle mixer reactor, or the like. Examples of reactor 10 are disclosed in pending U.S. application Ser. No. 16/289,296, filed Feb. 28, 2019 published as US Patent Publication No. ______ on ______, which is incorporated herein by specific reference in its entirety.

Biological suspension 12 includes cells or microorganisms and a growth medium in which the cells or microorganisms are suspended and grown. By way of example and not by limitation, reactor 10 can be used in culturing bacteria, fungi, algae, plant cells, animal cells, protozoans, nematodes, and the like. Examples of some common biologics that are grown include E. coli, yeast, bacillus, and CHO cells. In one embodiment, the biological suspension that is processed herein can be blood free, i.e., free of blood components such as plasma, red blood cells, white blood cells or platelets. Thus, the processed cells can be non-blood component cells. Reactor 10 can accommodate cells and microorganisms that are aerobic or anaerobic and are adherent or non-adherent. The composition for the medium is known in the art and changes based upon the cells or microorganisms being grown and the desired end product. In some uses, reactor 10 is used primarily only to grow and recover cells for subsequent use (e.g., preparing vaccine materials from the cells themselves). However, in many uses, the ultimate purpose of growing cells in reactor 10 is to produce and later recover biological products (such as recombinant proteins) that are exported from the cells into the growth medium. It is also common to use reactor 10 to grow cells in a master batch to prepare aliquots of cells for subsequent use as an inoculant for multiple subsequent batches of cells grown to recover biological products.

Although the disclosure herein is primarily designed for use with biological suspensions, the apparatus and methods of the present disclosure can also be used with non-biological suspensions where it is desired to separate solids from liquids. Such applications can be found in the production of chemicals, medicines, and other products. Accordingly, the discussions and examples set forth herein of separating a biological suspension and harvesting the separated components are also applicable to and should be considered as disclosure for separating non-biological suspensions and harvesting the separated components thereof.

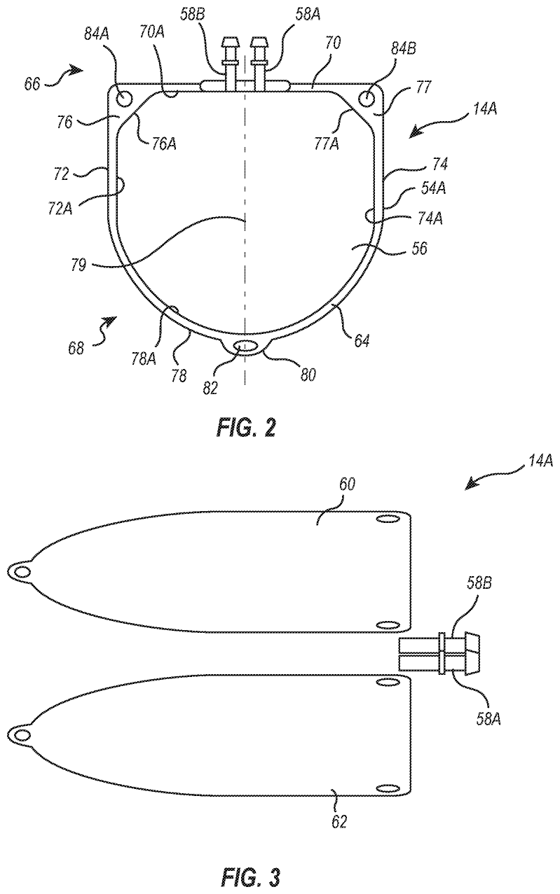

Once suspension 12 has been sufficiently grown in reactor 10 or otherwise produced, suspension 12 is dispensed into a bag assembly 14 (such as 14A or 14B). Depicted in FIG. 2 is one embodiment of a bag assembly 14A that comprises a flexible, collapsible bag 54A bounding a compartment 56. Bag assembly 14A further comprises a first port 58A and a second port 58B coupled to bag 54A and communicating with compartment 56. As depicted in FIG. 3, bag 54A is comprised of a first sheet 60 overlying a second sheet 62. Sheets 60 and 62 are bonded together (as shown in FIG. 2) to form a seam line 64 that encircles compartment 56. Seam line 64 can be produced by using conventional welding techniques such as heat welding, RF energy, ultrasonic, and the like. Other conventional techniques, such as by using an adhesive, can also be used to form seam line 64. Ports 58A and 58B are bonded between sheets 60 and 62 so as to form a sealed engagement therebetween. Ports 58A and 58B can also be bonded to sheets 60 and 62 by welding, adhesive or other conventional techniques. Although two ports 58A and 58B are shown, other numbers of ports, such as one, three, four or more ports, could be secured between sheets 60 and 62 so as to communicate with compartment 56. In other embodiments, as discussed below in more detail, ports 58A and 58B could be eliminated and one, two, three or more sections of tubing could be secured between sheets 60 and 62 so as to communicate with compartment 56.

First sheet 60 and second sheet 62 can comprise a flexible, water impermeable polymeric film such as a low-density polyethylene. The polymeric film can have a thickness that is at least or less than 0.02 mm, 0.05 mm, 0.1 mm, 0.2 mm, 0.5 mm, 1 mm, 2 mm, 3 mm or in a range between any two of the foregoing. Other thicknesses can also be used. The film is sufficiently flexible that it can be rolled into a tube without plastic deformation and can be folded over an angle of at least 90°, 180°, 270°, or 360° without plastic deformation.

The film can be comprised of a single ply material or can comprise at least two, three, four or more layers that are either sealed together or separated to form a multi-wall container. Where the layers are sealed together, the material can comprise a laminated or extruded material. The laminated material comprises two or more separately formed layers that are subsequently secured together by an adhesive. One example of an extruded material that can be used in the present disclosure is the Thermo Scientific CX3-9 film available from Thermo Fisher Scientific. The Thermo Scientific CX3-9 film is a three-layer, 9 mil cast film produced in a cGMP facility. Although Sheets 60 and 62 can also be formed from a five-layer cast film CX5-14 available from Thermo Fisher Scientific, more favorable results have typically been obtained by forming bag 54A from a three layer film. This is because bags 54A formed from a three layer film are more flexible than bags 54A formed from a five layer film and, as a result, produce fewer creases or folds during centrifugation. Such creases or folds in bags 54A can have a negative influence during centrifugation because, in part, they can restrict movement of portions of suspension 12. Accordingly, sheets 60 and 62 are commonly formed from extruded or laminated films having between 2-4 layers and, more commonly three layers and having a thickness between 7 mil and 11 mil and more commonly between 8 mil and 10 mil.

The material can be approved for direct contact with living cells and be capable of maintaining a solution sterile. In such an embodiment, the material can also be sterilizable such as by ionizing radiation. Examples of materials that can be used in different situations are disclosed in U.S. Pat. No. 6,083,587 which issued on Jul. 4, 2000 and United States Patent Publication No. US 2003-0077466 A1, published Apr. 24, 2003, which are hereby incorporated by specific reference.

Bags 54A are commonly sized so that compartment 56, when and if inflated, has a volume of at least or less than 0.5 liters, 1 liter, 1.5 liters, 2 liters, 2.5 liters, 3 liters, 5 liters, 6 liters, 10 liters, 13 liters, 15 liters or in a range between any two of the foregoing values. Other volumes can also be used.

Returning to FIG. 2, bag 54A has a top end 66 where ports 58 are disposed and an opposing bottom end 68. Seam line 64 comprises a top seam line section 70 disposed at top end 66 to which ports 58A and 58B are connected and which has an linear inner edge 70A. Seam line 64 also includes opposing side seam line sections 72 and 74 that include linear inner edges 72A and 74A, respectively, that are perpendicular to inner edge 70A. A corner seam line section 76 extends at an angle between top seam line section 70 and side seam line section 72 having a linear inner edge 76A while a corner seam line section 77 extends between top seam line section 70 and side seam line section 74 having a linear inner edge 77A. Inner edges 76A and 77A intersect with inner edge 70A to each form an inside angle therebetween that is in a range between 110° and 170° with between 130° and 150° being more common. Other angles can also be used. In other embodiments corner seam line sections 76 and 77 can be configured to that inner edges 76A and 77A are curved. In yet other embodiments, corner seam line sections 76 and 77 can be eliminated and top seam line section 70 can intersect directly with side seam line sections 72 and 74.

Finally, seam line 64 also includes a bottom seam line section 78 disposed at bottom end 68 that has an inner edge 78A that arches away from top seam line section 70 and that extends as a smooth continuous curve between opposing side seam line sections 72 and 74. The curve of bottom seam line section 78 can be an arc, U-shape, segment of an oval, segment of an ellipse, or have other configurations. In one embodiment, bottom seam section line 78 comprises at least 20%, 25%, 30%, 35%, or 40% of the entire length of seam line 64 that encircles compartment 56. As depicted and in view of the foregoing, it is appreciated that top end 66 and bottom end 68 of bag 54A, and particularly the seam lines thereat, have different configuration, i.e., they are not symmetrical about a lateral axis that extends between side seam line section 72 and 74.

More specifically, with regard to a central longitudinal axis 79 that extends between top seam line section 70 and bottom seam line section 78, compartment 56 is more constricted about central longitudinal axis 79, i.e., compartment 56 is narrower, at bottom end 68 than at top end 66. The constricting of compartment 56 at bottom end 68 functions to help consolidate the pellet produced during centrifugation, as discussed below, at a central location within compartment 56. Consolidating the pellet in a constricted area makes the pellet thicker with more mass so that the pellet is more stable and less likely to break apart. Consolidating the pellet into a constricted area also assists with the removal of the supernatant and can assist with subsequent removal of the pellet that is located in a smaller area. Although inwardly tapering bottom end 68 of bag 54A achieves the above discussed added benefits, in other embodiments bag 54A could be formed so that bottom end 68 does not taper or does not taper more than top end 66. That is, top end 66 and bottom end 68 of bag 54A, and particularly the seam lines thereat, could be symmetrical about a lateral axis that extends between side seam line section 72 and 74.

Bag 54A further comprises a hanging tab 80 centrally formed at bottom end 68 with an opening 82 extended therethrough. Openings 84A and 84B also extend through sheets 60 and 62, such as through seam line 64, at opposing sides of bag 54A at top end 66. Openings 82 and 84 can be used for hanging or supporting each bag assembly 14 in a vertically up orientation or vertically down orientation.

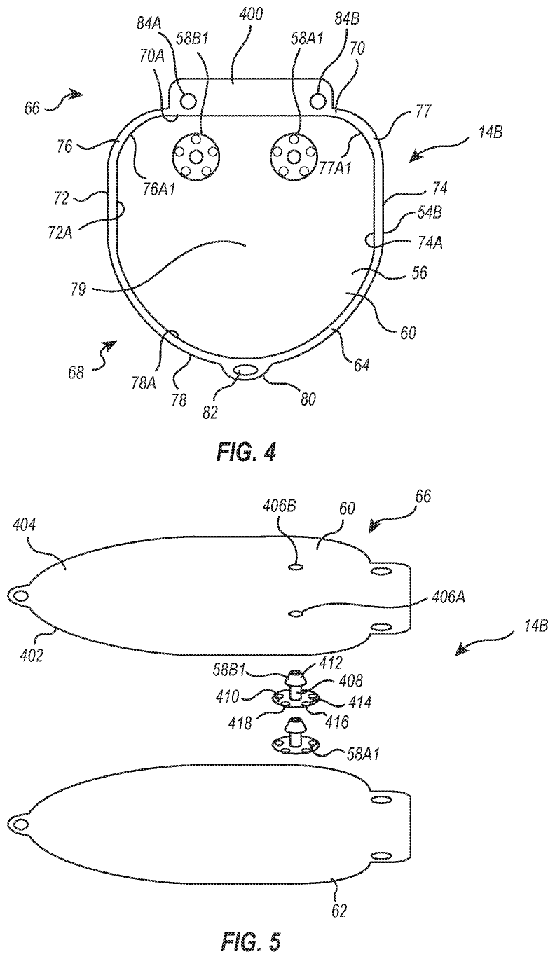

It is appreciated that bag assembly 14 can also have a variety of other configurations. For example, depicted in FIGS. 4 and 5 is a bag assembly 14B. Bag assembly 14B comprising a bag 54B having ports 58A1 and 58B1 mounted thereon. Bag 54B has substantially the same structural elements and substantially the same configuration as bag 54A and can be made of the same material as bag 54A. As such, like elements between bags 54A and 54B are identified by like reference characters and the prior discussion with regard to the elements of bag 54A is also applicable to bag 54B. Bag 54B differs from bag 54A in that corner seam lines sections 76 and 77 are curved. As such, corner seam line section 76 has an inner edge 76A1 that inwardly curves in an arc from inner edge 72A to inner edge 70A and corner seam line section 77 has an inner edge 77A1 that inwardly curves in an arc from inner edge 74A to inner edge 70A. In other embodiments, inner edges 76A1 and 77A1 could also be linear as in bag 54A. Bag 54B also has a hanging tab 400 formed at top end 66 and outwardly projecting from top seam line section 70. Openings 84A and 84B extend through hanging tab 400 and are used for supporting bag 54B in a vertical orientation. Hanging tabs 80 and 400 can simply comprise portions of the flexible film used to bound compartment 56 of bag 54B.

Bag assembly 14B differs from bag assembly 14A in that bag assembly 14B does not include ports 58A and 58B (FIG. 2) projecting from top seam line section 70. Rather, bag assembly 14B includes ports 58A1 and 58B1 that are secured to and outwardly project from first sheet 60 at or toward top end 66 at locations spaced apart from seam line 64. Specifically, first sheet 60 has an inside face 402 and an opposing outside face 404 having opening 406A and 406B extending therethrough at locations spaced apart from the perimeter edge of first sheet 60. Each port 58A1 and 58B1 includes a tubular stem 408 having an annular flange 410 outwardly projecting from one end and an annular barb 412 outwardly projecting from an opposing end. Flange 410 has a top side 414 that faces toward stem 408 and an opposing bottom side 416. One or more projections 418 can project from bottom side 416. Projections 418 ensure that a spacing is formed between first sheet 60 and second sheet 62 at ports 58A1 and 58B1 so that fluid can freely flow from compartment 56 out through stems 408. During assembly, stems 408 of ports 58A1 and 58B1 are passed though openings 406A and 406B, respectively, and flanges 410 are secured, such as by welded, adhesive or the like, to inside face 402 of first sheet 60. Ports 58A1 and 58B1 can then be used in the same way as discussed herein with regard to ports 58A and 58B.

In the assembled configuration, ports 58A1 and 58B1 thus extend through first sheet 60 as opposed to simply being secured between sheets 60 and 62. Furthermore, ports 58A1 and 58B1 are typically, though not required, equally spaced on opposing sides of central longitudinal axis 79 and are disposed at top end 66 at locations spaced apart from seam line 64. With reference to bag assembly 14B and longitudinal axis 79 being vertically orientated, as shown in FIG. 4, ports 58A1 and 58B1 are typically located within the upper ⅓, ¼, or ⅕ of the area of outside face 404 of first sheet 60 or within the upper ⅓, ¼, or ⅕ of the height/length of first sheet 60/bag 54B. Securing ports 58A1 and 58B1 on the face of first sheet 60, as discussed above, results in less leaking, less integrity testing, and easier attachment than ports 58A and 58B welded between first sheet 60 and second sheet 62. However, securing ports 58A1 and 58B1 on the face of first sheet 60 can make it more difficult to remove all of the fluid from bag 54B relative to having ports 58A and 58B welded between sheets 60 and 62. As such, the selected configuration for bag assemblies 14 can depend on the intended use.

In the above discussed embodiments, bags 54 (such as 54A or 54B) are disclosed as being two-dimensional, pillow type bags formed by seaming together two overlapping sheets of flexible film. In other embodiments, however, bags 54 can comprise three-dimensional bags that are typically formed by seaming together three, four or more sheets of flexible film. In yet another embodiment, bags 54 can be blown bags that are blown from a polymeric material and have no seam lines except at the opening through which they are blown. Because of the material that is used to form bags 54, which includes bags 54A and 54B and the other alternatives discussed herein, bags 54 are collapsible in that they can be fully inflated and fully deflated flat without plastic deformation. Bags 54 can also be folded over or rolled into a tube without plastic deformation.

Returning to FIG. 1, an inlet line 90 fluid couples reactor 10 to bag 54A.

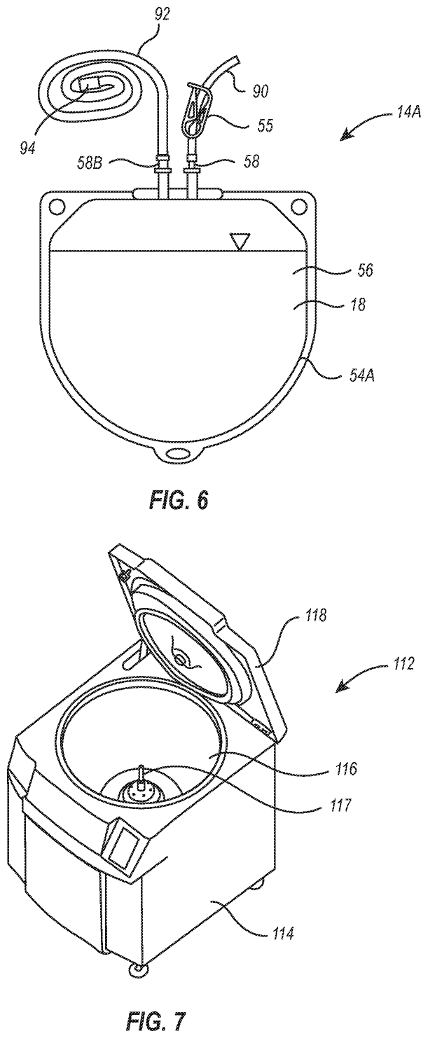

Specifically, inlet line 90 is fluid coupled with port 58A disposed on bag 54A. A clamp 55 is mounted on inlet line 90. Clamp 55 can be manually adjusted to regulate the flow of suspension 12 through inlet line 90 and can seal off inlet line 90 to prevent fluid flow therethrough. In addition, an outlet line 92 can be coupled with port 58B disposed on bag 54A. As depicted in FIG. 6, outlet line 92 has a terminal end with a fitting 94 disposed thereat. Fitting 94 can comprise a cap that seals outlet line 92 closed or it can comprise an aseptic connector that maintains outlet line 92 sealed closed but enables outlet line 92 to be selectively fluid coupled with another line under aseptic conditions. Other fittings can also be used. In yet other embodiments, fitting 94 can be eliminated and the terminal end of outlet line 92 can simply be sealed closed, such as by being welded closed.

Inlet line 90 and outlet line 92 can likewise be attached to ports 58A1 and 58A2 (FIG. 4) of bag 54B. It is noted that securing ports 58A1 and 58B1 on the face of first sheet 60 of bag 54B, as opposed to welding ports 58A and 58B between first sheet 60 and second sheet 62 (FIG. 3), makes it easier to store bag assembly 14B with lines 90 and 92 within an insert, bucket, and/or rotor of a centrifuge with decreased risk of line 90 or 92 coming out of the insert, bucket, and/or rotor during centrifugation. That is, because lines 90 and 92 project horizontally off of ports 58A1 and 58B1 into the insert, bucket and/or rotor, as opposed to projecting vertically upward, lines 90 and 92 are more easily placed and retained within the insert, bucket and/or rotor. Further information with regard to using bag assemblies 14 within an insert, bucket, and/or rotor of a centrifuge is disclosed in U.S. application Ser. No. 16/289,296 (US Patent Publication No. ______), which was previously incorporated by reference.

Once bag 54A has been filled with a desired amount of suspension 12, a portion of inlet line 90 upstream of clamp 55 is sealed closed and then cut, thereby separating bag 54 (such as 54A or 54B) from reactor 10. Accordingly, as depicted in FIG. 6, bag assembly 14A can be further defined as comprising a portion of inlet line 90, clamp 55, outlet line 92 and, if used, fitting 94. Each of the different elements of each bag assembly 14 can be modified, eliminated or replaced. For example, different numbers of ports 58, such as 1, 3, 4 or more, can be coupled with bag 54 with a separate fluid line coupled with each port. In other embodiments, one or more of ports 58 can be eliminated and the corresponding fluid lines can be coupled directly to bag 54. Likewise, although different embodiments have different advantages, bag assemblies 14A, 14B, and the other embodiments disclosed herein can typically be interchangeably used. Other shapes and volumes of bag assemblies 14 and bags 54 can also be used. Other examples of bag assemblies and bags and other systems and methods for filling the bags are disclosed in U.S. application Ser. No. 16/289,296 (US Patent Publication No.), which was previously incorporated by reference. For example, as discussed in detail in the '296 application, multiple bag assemblies 14 can be concurrently coupled in parallel or series to reactor 10 through a manifold. Fluid flow to each bag assembly 14 can be controlled by regulating the fluid flow through the manifold. Once a bag assembly 14 is filled, it can be sealed and cut from the manifold and processed as described herein.

Compartment 56 of each bag assembly 14 is sterile as suspension 12 is first delivered therein and inlet line 90 provides a sterile fluid pathway through which suspension 12 can be delivered into compartment 56. Reactor 10, inlet line 90 and bag assemblies 14 combine to form a closed system in that the internal area they bound is not exposed to the open environment. As used in the specification and appended claims, the terms “sterile” and “sterilized” mean that the related item has been subjected to a sterilization process so that the sterility assurance level (SAL) is 10⁻⁶ or lower. Sterility assurance level (SAL) is the probability that a single unit that has been subjected to sterilization nevertheless remains non-sterile, i.e., is not free from bacteria or other living microorganisms. As such, an SAL of 10⁻⁶ means that there is a 1 in 1,000,000 chance that a unit subjected to the sterilization process remains non-sterile.

As noted above, after bag assemblies 14 are filled to their desired amount with suspension 12 and clamp 55 is closed, a section of inlet line 90 upstream of clamp 55 is welded closed. Inlet line 90 is then cut at a central location along the welded section so as to sever bag assembly 14 from reactor 10 as depicted in FIG. 6. By cutting inlet line 90 at a central location along the welded section, no leaking of suspension 12 occurs from inlet line 90. In contrast to welding and cutting inlet line 90 upstream of clamp 55, inlet line 90 could alternatively be welded at a location between clamp 55 and bag 54 and then cut through the welded section. This approach would eliminate clamp 55 as being retained as part of the bag assembly.

Suspension 12 can be dispensed into bag assembly 14 at different times during the growth cycle. For example, in one method, once suspension 12 has reached a desired growth stage, all of suspension 12 within reactor 10 can be dispensed into one or more bag assemblies 14 for further processing. Alternatively, portions of suspension 12 within reactor 10 can be dispensed into bag assemblies 14 at spaced apart intervals during the growth cycle, e.g., at days 14, 16, 18, etc. In this method, reactor 10 can be replenished with fresh medium to compensate, either exactly or approximately, for the volume of suspension 12 removed from reactor 10. This method may be appropriate during bioproduction cell culture process development to determine any variability in cell performance or the production protein characteristics resulting from extended run time.

Next, separated bag assembly 14 is moved to a centrifuge for separation of suspension 12 therein. For example, depicted in FIG. 7 is a centrifuge 112. Centrifuge 112 is depicted as a floor standing centrifuge. However, centrifuge 112 can comprise any type, shape, or configuration of centrifuge. In general, centrifuge 112 has a body 114 that bounds a cavity 116 and has a spindle 117 disposed therein. Spindle 117 is rotated by a motor disposed within body 114. A lid 118 can be hingedly mounted or removably secured to body 114 for selectively covering cavity 116 during operation. Cavity 116 is configured to receive a rotor that couples with spindle 117 and is rotated within cavity 116 by the rotation of spindle 117. The rotor is configured to receive and support one or more bag assemblies. Floor standing centrifuges are commonly used because they have an enlarged cavity 116 that enables handling larger and/or more bag assemblies 14 during each run or operational cycle of the centrifuge. However, a table top centrifuge can also be used.

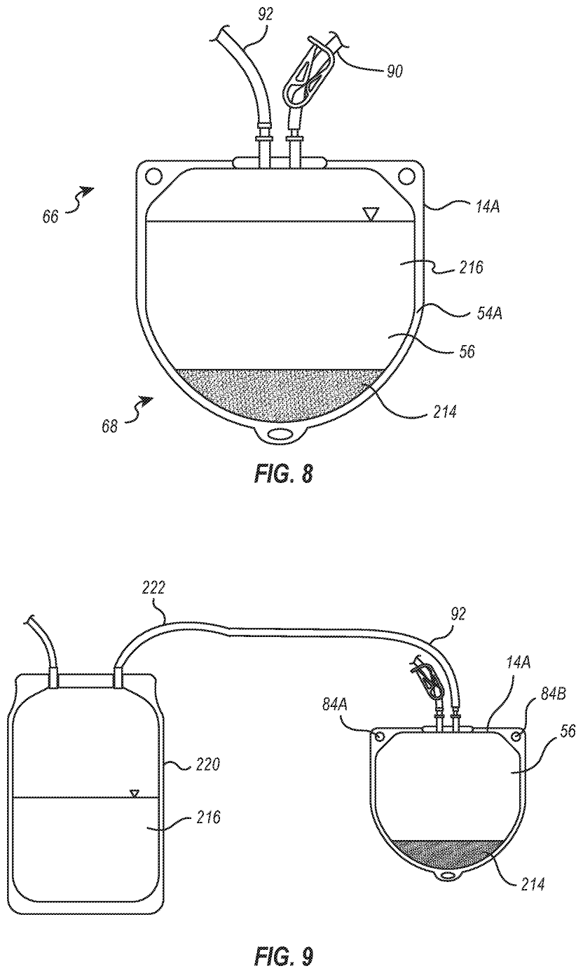

As bag assembly 14 is rotated within centrifuge 112, the centrifugal force caused by spinning of the rotor of centrifuge 112 causes at least a portion of the solids within suspension 12, e.g., the cells, microorganisms, and/or other solids, to sediment out of the solution and collect within bottom end 68 of bag assembly 14A to form a pellet 214, as shown in FIG. 8. The remaining fluid collects as a supernatant 216 above pellet 214 and can include some solids. Pellet 214 has a density that is greater than the density of supernatant 216. Pellet 214 can also have a viscosity that is greater than the viscosity of supernatant 216. For example, the density and viscosity of pellet 214 can be at least 2, 5, 7, 10, 15, 30 or 50 times that of supernatant 216. In one application, pellet 214 can comprise a paste or a slurry while supernatant 216 typically comprises a free flowing liquid, like water. Methods, systems and alternatives for centrifugally rotating bag assemblies 14/bags 54 so as to form pellet 214 and supernatant 216 are disclosed in U.S. application Ser. No. 16/289,296 (US Patent Publication No. ______), which was previously incorporated by reference.

As discussed in U.S. application Ser. No. 16/289,296 (US Patent Publication No. ______), there are benefits to having the bag assembly and centrifuge rotor configured so that pellets 214 form and consolidate at one location at (or near) bottom end 68 of bag assembly 14. Some cells, microorganisms, and/or other solids of suspension 12 are found to make a generally firm and compact pellet 214 that is not easily disturbed and resuspended into supernatant 216. In contrast, however, other cells such as mammalian cells, like Chinese hamster ovary (CHO) cells, can form a slurry or very loose pellet 214 and thus are easily resuspended into supernatant 216. The time and speed at which bag assemblies 14 containing suspension 12 are rotated by centrifuge 112 is dependent in part on the composition and volume of suspension 12. However, bag assemblies 14 are typically spun at a rate between 300 rpm and 5,000 rpm or between 300 rpm and 7,000 rpm with between 2,000 rpm and 5,000 rpm being more common. The time of rotation is typically between 5 minutes and 90 minutes with between 5 minutes and 30 minutes being more common. Other rates and times can also be used.

Once suspension 12 within bag assemblies 14 is separated into pellets 214 and supernatant 216, the next step is to remove supernatant 216 from bag assemblies 14. Where pellet 214 is firm and not easily resuspended, as discussed above, this step can be accomplished by fluid coupling outlet line 92 to a container 220 as depicted in FIG. 9. For example, where fitting 94 (FIG. 6) is a cap, outlet line 92 can be cut, such as in a laminar flow hood, and then fluid coupled with container 220 using a sterile connection. In one method, outlet line 92 can be welded to an inlet line 222 extending from container 220 or can be directly coupled to container 220. Alternatively, where fitting 94 (FIG. 6) is an aseptic connector, fitting 94 can simply be coupled to a corresponding aseptic connector on inlet line 222 of container 220 to form a sterile fluid coupling. Other methods of fluid coupling can also be used.

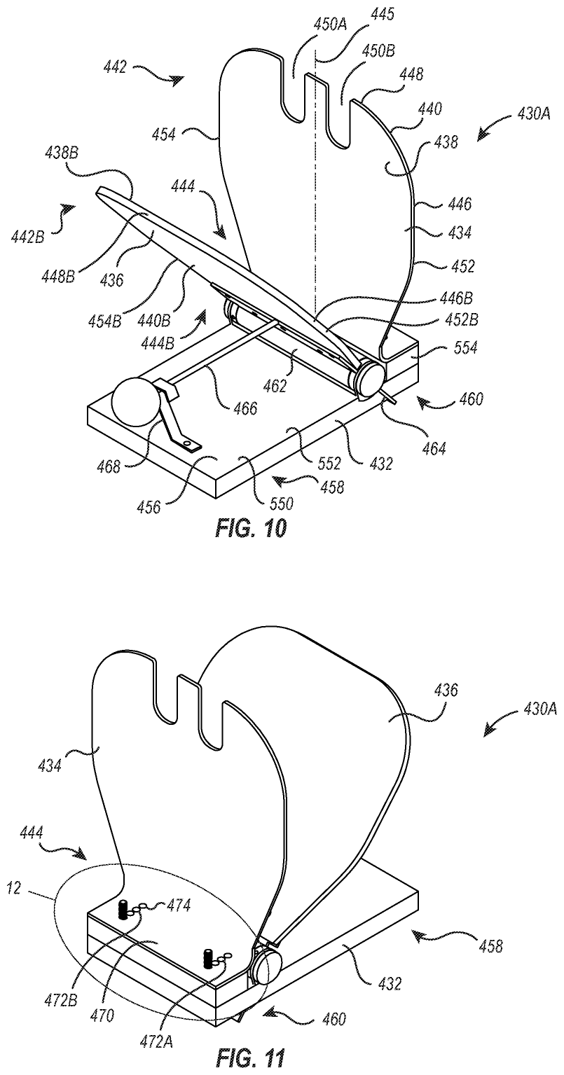

In one method of separating supernatant 216 from pellet 214, an expressor can be used to drive supernatant 216 from bag assembly 14 and into container 220 so that pellet 214 remains within bag 54. As used in the specification and appended claims, the term “expressor” is broadly intended to comprise any type of device that can mechanically compress bag assembly 14 for driving supernatant 216 therefrom. For example, depicted in FIG. 10 is one embodiment of an expressor 430A incorporate features of the present disclosure. In general, expressor 430A comprises a base 432 having a first platen 434 coupled thereto. A second platen 436 is coupled to base 432 so that second platen 436 can selectively move toward and away from first platen 434. In the depicted embodiment, base 432 has a top surface 456 extending between a first end 458 and an opposing second end 460.

First platen 434 is depicted as comprising a plate having an inside face 438 and an opposing outside face 440 that both extend between an upper end 442 and an opposing lower end 444. First platen 434 has a perimeter edge 446 having a tapered configuration similar to the tapered configuration of bag assemblies 14. More specifically, perimeter edge 446 has an upper edge portion 448 having two spaced apart notches 450A and 450B centrally recessed thereon. More specifically, notches 450A and 450B are recessed into perimeter edge 446 at upper edge portion 448 so as to extend toward lower end 444 and pass through first platen 434 between inside face 438 and opposing outside face 440.

In one embodiment, first platen 434 has a central longitudinal axis 445 extending between upper end 442 and an opposing lower end 444 that is centrally disposed between notches 450A and 450B. Each notches 450A and 450B typically has a width W of at least 0.5 cm, 0.75 cm. 1 cm, 1.5 cm or 2 cm or is in a range between any two of the foregoing values. Notch 450A and 450B are sized so that when bag assembly 14B (FIG. 4) is used with expressor 430A, first sheet 60 can be disposed against inside face 438 of first platen 434 with ports 58A1 and 58B1 being received within notches 450A and 450B, respectively. This configuration enables all or substantially all of bag 54B to be uniformly compressed between platens 434 and 436, as discussed further below. The number and location of notches 450 can vary based upon the number and location of ports 58 disposed on first sheet 60. For example, if one, three or more ports 58 are used, then one, three or more notches 450 can be formed. Furthermore, is no ports 58 on disposed on first sheet 60, such as when bag assembly 14A (FIG. 2) is used, notches 450 can be eliminated.

Perimeter edge 446 of first platen 434 also includes opposing side edge portions 452 and 454 that extend between upper end 442 and opposing lower end 444. Side edge portions inwardly taper at lower end 444 relative to upper end 442 or, expressed in other terms, side edge portions outwardly taper at upper end 442 relative to lower end 444. As a result, first platen 434 has a wider width at upper end 442 than at lower end 444. In one embodiment, the width of first platen 434 at upper end 442 can be greater than the width of base 432. The opposing side edge portions 452 and 454 of first platen 434 are tapered so that first platen 434 has a configuration complementary to tapered bag 54, as discussed above. As a result, first platen 434 uniformly supports all of one side of bag 54 so that bag 54 can be uniformly compressed between platens 434 and 436, as discussed below. In other embodiments, first platen 434 need not be tapered but could have a substantially constant width along the length. However, were bags 54 are tapered, forming platens with a complementary tapers reduces material cost and maximizes uniform compression.

First platen 434 is secured to base 432 at or toward second end 460. In one embodiment, first platen 434 is secured so that when base 432 and/or top surface 456 thereof are horizontally disposed, inside face 438 is vertically disposed, i.e., is orthogonal to base 432 and/or top surface 456. In other embodiments, first platen 434 can be angled so that an angle is formed between top surface 456 of base 432 and inside face 438 of first platen 434 that is at least or less than 110°, 120°, 130°, 140°, 150° or 160° or is in a range between any two of the foregoing angles.

Second platen 436 has substantially the same configuration as first platen 434. As such, like elements between platens 436 and 434 are identified by like reference characters except that the reference characters for second platen 436 include the suffix “B.” For example, second platen 436 comprises a plate having an inside face 438B and an opposing outside face 440B that both extend between an upper end 442B and an opposing lower end 444B. Second platen 436 has a perimeter edge 446B having a tapered configuration similar to the tapered configuration first platen 434, i.e., similar to the tapering of bag assemblies 14/bags 54. More specifically, perimeter edge 446B has an upper edge portion 448B and two opposing side edge portions 452B and 454B. Side edge portions 452B and 454B inwardly taper at lower end 444B relative to upper end 442B or outwardly taper at upper end 442B relative to lower end 444B.

Second platen 436 distinguishes from first platen 434 in that second platen 436 does not include notches 450A and 450B. However, in an alternative embodiment, the one or more notches 450A and 450B could be formed on second platen 436 and not on first platen 434. In this design, bag assembly 54B (FIG. 4) would be positioned so that first sheet 60 is disposed against second platen 436 and ports 58A1 and 58B1 are again received within notches 450A and 450B, respectively.

To enable visual inspection of bag assemblies 14 during operation of expressor 430A, as discussed below, second platen 436 is commonly formed from a transparent polymer such as acrylic or polyethylene terephthalate glycol. Although first platen 434 could also be made from a transparent polymer, it is commonly less useful. As such, first platen 434 is commonly made from an opaque material, such as an opaque plastic or a metal.

Second platen 436 is movably mounted to base 432 so that second platen 436 can selectively move toward and away from first platen 434. More specifically, second platen 436 is movable between a retracted position, as shown in FIG. 10, where second platen 436 is moved away from first platen 434, and a collapsed position, as shown in FIG. 13, where second platen 436 is moved toward first platen 434. Returning to FIG. 10, in one embodiment a hinge 462 is mounted on base 432, such as by being secured to top surface 456. Lower end 444B of second platen 436 is secured to hinge 462 so that second platen 436 can hingedly pivot relative to base 432 between the retracted position and the collapsed position. It is appreciated that hinge 462 can have a variety of different configurations and can comprise two or more separate hinges the hingedly secure second platen 436 to base 432.

As shown in FIG. 10, base 432 includes a platform 550 having a top surface 552 that extends between opposing ends 458 and 460. A riser 554 is disposed on top of platform 550 at second end 460 with first platen 434 being disposed on top of riser 554. Riser 554 has a height that corresponds to the height of hinge 462. As such, when second platen 436 is in the collapsed position (FIG. 13), platens 434 and 436 are disposed at substantially the same elevation, i.e., platens 434 and 436 are aligned horizontally. This alignment helps to ensure that bag assembles 14 are uniformly compressed between platens 434 and 436. It is appreciated that the alignment of platens 434 and 436 can be achieved in a variety of other ways. For example, riser 554 could be eliminated and hinge 464 could be recessed within a slot formed on platform 550. It is also noted that riser 554 and platform 550 can be formed as a single, unitary, integral structure as opposed to two parts connected together.

In one embodiment of the present disclosure, means are provided for mechanically moving second platen 436 toward first platen 343, i.e., for mechanically moving second platen 436 from the retracted position to the collapsed position. By way of example, a spring 464 is coupled with hinge 462 so as to resiliently urge or bias second platen 436 toward the collapsed position, i.e., toward first platen 434. In alternative embodiments, the means can comprise other convention drive mechanism such as a pneumatic or hydraulic piston, a gear assembly, screw drive, worm drive or linkage driven by a motor or compressor. Other spring or elastic band configurations could also be used. For example, one or more elastic bands could extend between platens 434 and 436 to resiliently urge second platen 436 toward the collapsed position.

As discussed below in more detail, the use of spring 464 has the advantage that it is inexpensive and does not require the use of a motor or controller. Other embodiments, such as the use of a piston or motor driven driver can have the advantage that they can be more precisely controlled. For example, the amount, rate and time that a force is applied to second platen 436 can be precisely controlled through a controller.

An elongated handle 466 projects from outside face 440B of second platen 436 at lower end 444B. A catch 468 is disposed on base 432 at or toward first end 458. Handle 466 is used to manually pivot second platen 436 to the retracted position. Catch 468 can then engage handle 466 to hold second platen 436 in the retracted position. When handle 466 is released from catch 468, second platen 436 resiliently rebounds under the force of spring 464 toward the collapsed position.

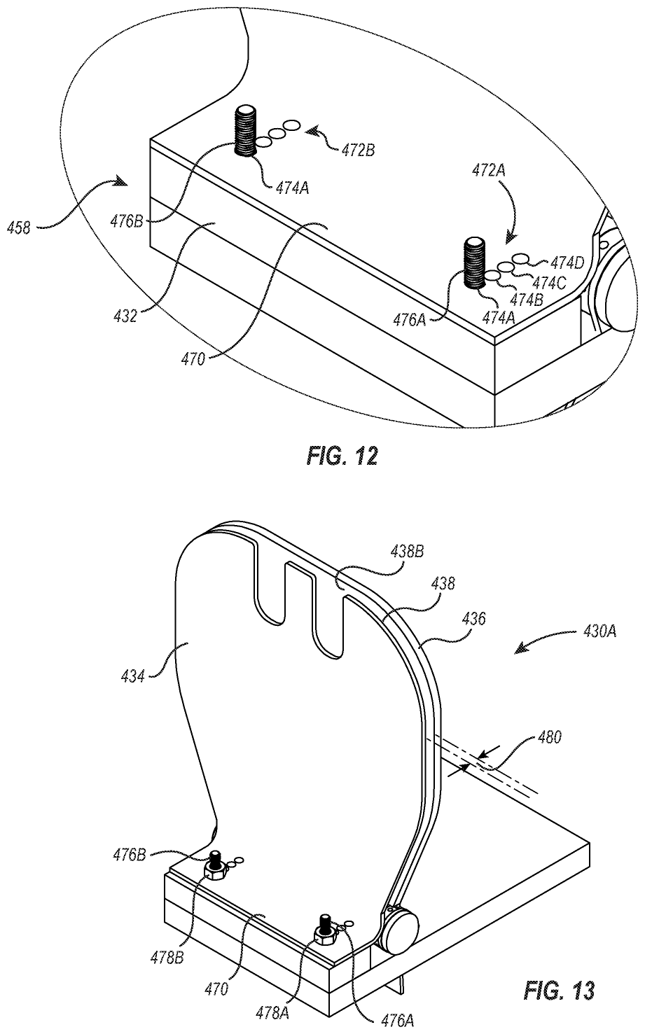

In one embodiment, first platen 434 can be permanently secured to or integrally formed with base 432. However, in the present embodiment, first platen 434 is adjustably mounted to base 432 so that a gap spacing formed between first platen 434 and second platen 436 can be adjusted. For example, as shown in FIG. 11, a foot 470 outwardly projects from first platen 434 at lower end 444 so as to extend away from second platen 436. In one embodiment, foot 470 can extend orthogonal to first platen 434. Extending through foot 470 are two spaced rows 472A and 472B of a plurality of holes 474. Rows 472A and 472B are in parallel alignment and extend orthogonal to first platen 434. As better shown in FIG. 12, each row 472A and 472B is shown as comprising aligned holes 474A, 474B, 474C and 474D. Other numbers of holes 474, such as at least 2, 3, 4, or 5, can also be used. In the depicted embodiment, each hole 474 is round. However, other configurations can also be used.

Upwardly projecting from base 432 at second end 458 are a pair of spaced apart mounting shafts 476A and 476B that are threaded. Mounting shafts 476 are configured to pass through holes 474. For example, as depicted in FIG. 12, mounting shafts 476A and 476B are received within holes 474A of rows 472A and 472B, respectively. In turn, nuts 478A and 478B, as shown in FIG. 13, can be threaded onto mounting shafts 476A and 476B, respectively, so as to bias against foot 470, thereby securely fixing first platen 434 to base 432. With first platen 434 so positioned and second platen 436 moved to the collapsed position, as shown in FIG. 13, a gap spacing 480 is formed between first platen 434 and second platen 436.

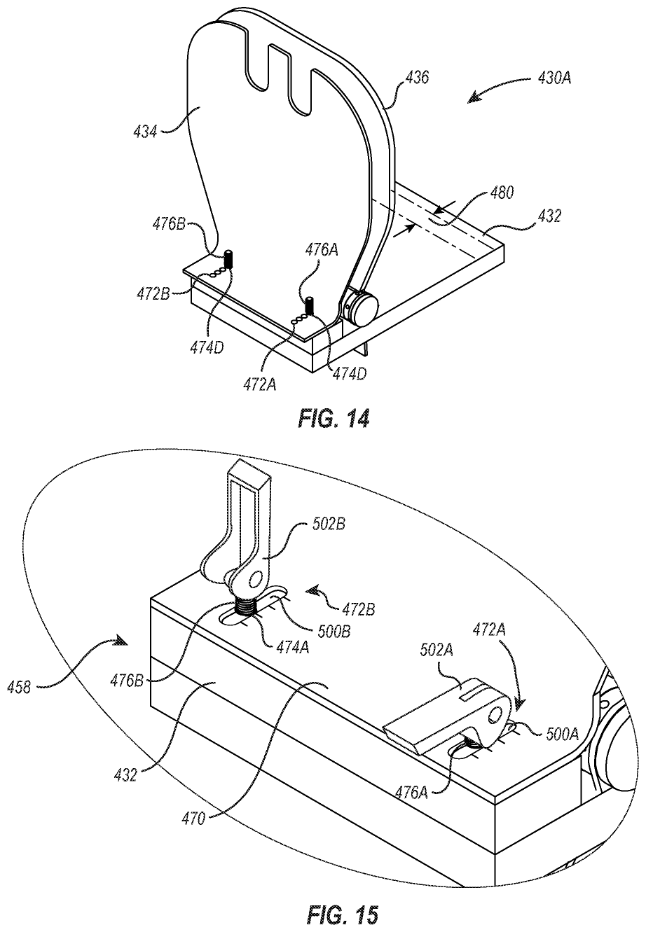

More specifically, hinge 462 is configured so that second platen 436 can only rotate to a fixed orientation before it is mechanically stopped at the collapsed position. The fixed orientation for stopping second platen 436 is typically when inside faces 438 and 438B of platens 434 and 436 are disposed in parallel alignment. Thus, by spacing apart first platen 434 and second platen 436, gap spacing 480 is formed between inside face 438 of first platen 434 and inside face 438B of second platen 436 when second platen is in the collapsed position. When the fixed orientation for stopping second platen 436 is set so that platens 434 and 436 are disposed in parallel alignment, the gap spacing 480 can be uniform along the lengths of platens 434 and 436. This has the benefit in that bag 54 is uniformly compressed between platens 434 and 436. However, in other embodiments, the fixed orientation for stopping second platen 436 can be set so that platens 434 and 436 are slightly angled relative to each other. For example, inside faces 438 and 438B of platens 434 and 436 can be disposed in converging planes having an inside angle in a range between 1° and 15° with between 1° and 10° and between 1° and 5° being more common. In these embodiments, the gap spacing 480 can vary along the lengths of platens 434 and 436 when second platen 436 is in the collapsed position. Thus, gap spacing 480 referenced herein can refer to a minimum gap spacing or a maximum gap spacing.

Where it is desired to increase the width of gap spacing 480, nuts 478 can be removed and first platen 434 raised vertically off of mounting shafts 476. First platen 434 can then be repositioned so that mounting shafts 476 are positioned within one of the other holes 474B-474D of foot 470 that are closer to first platen 434. For example, as shown in FIG. 14, mounting shafts 476 are now placed within holes 474D of rows 472A and 472B. Nuts 478 (FIG. 13) can again be threaded onto mounting shafts 476 so as to bias against foot 470 and thereby securely fix first platen 434 to base 432. In this second position of first platen 434, gap spacing 480 when second platen 436 is in the collapsed position is now larger than when first platen 434 was in the first position shown in FIG. 13. Accordingly, by selectively moving mounting shafts 476 to different holes 474, the width of gap spacing 480 can be selectively adjusted and set to a desired value. That is, gap spacing 480 is adjusted by laterally moving first platen 434 relative to base 432 and/or second platen 436. Such movement of the first platen 434 does not require pivoting or rotating of first platen 434.

In one embodiment, expressor 430A can be configured so that gap spacing 480 can be selectively adjusted by an amount of at least 0.5 cm, 1 cm, 2 cm, 3 cm or 4 cm or in a range between any two of the foregoing values. Furthermore, gap spacing 480 is typically at least 0.5 cm, 1 cm, 2 cm, 3 cm or 4 cm or is a range between any two of the foregoing values when second platen 436 is in the collapsed position. The benefit of being able to adjust and/or set the width of gap spacing 480 will be discussed below in greater detail.

The use of mounting shafts 476, holes 474 and nuts 478 is one example of means for selectively adjusting the width of gap spacing 480 between platens 434 and 436. However, it is appreciated that a variety of other mechanism can likewise be used to selectively adjust the width of gap spacing 480 between platens 434 and 436. By way of example and not by limitation, separate holes 474 of each row 472 could be replaced with elongated channels through which mounting shafts 476 can slide. Mounting shafts 476 and nuts 478 are also one example of fasteners that can be used to releasably secure first platen 434 to base 432. In other embodiments, mounting shafts 476 and nuts 478 can be replaced with bolts, screws or other fasteners that passed down through select holes 474 or channels formed on foot 470 and secure into base 432 for adjusting gap spacing 480. In yet other embodiments, mounting shafts 476 and nuts 478 can be replaced with one or more clamps, latches, catches, cams or other types of releasable fasteners for adjusting gap spacing 480.

FIG. 15 depicts one alternative embodiment of the means for selectively adjusting the width of gap spacing 480 between platens 434 and 436. In this embodiment, the rows of holes 474 have been replaced with elongated slots 500A and 500B through which shafts 476A and 476B pass, respectively. Cams 502A and 502B are rotatably mounted on shafts 476A and 476B, respectively. Cams 502A and 502B can be selectively rotated between a locked position (cam 502A) where the cam presses foot 470 against base 432 so as to secure first platen 434 relative to base 432 and an unlocked position (cam 502B) where foot 470 is released from base 432 so that first platen 434 can freely move relative to base 432. That is, with cams 502A and 502B in the unlocked position, first platen 434 can freely move relative to base 432 by sliding shafts 476 within elongated slots 500.

In the above discussed embodiments, gap spacing 480 is adjusted by moving first platen 434 relative to base 432/second platen 436. However, in an alternative embodiment, expressor 430A can be configured so that gap spacing 480 is adjusted by moving second platen 436 relative to base 432/first platen 434. For example, a foot could project from hinge 462 having holes or slots formed thereon. Any of the above discussed fasteners used with first platen 434 could then be used to releasably secure second platen 436 to base 432 at spaced apart locations along the length of base 432 so as to adjust the width of gap spacing 480 when second platen 436 is in the collapsed position. Thus, gap spacing 480 can also be adjusted by laterally moving second platen 436 relative to base 432 and/or first platen 434 without pivoting or rotating of second platen 436.

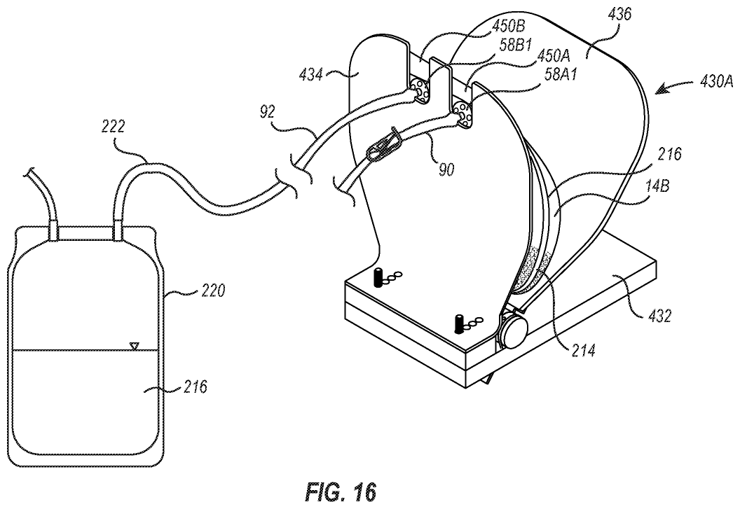

Expressor 430A can be used to drive supernatant 216 from bag assemblies 14 into container 220. For example, as depicted in FIG. 16, once suspension 12 within bag assembly 14B is separated into pellets 214 and supernatant 216, outlet line 92 of bag assembly 14B is fluid coupled to inlet line 222 of container 220 or can be directly coupled to container 220 using any conventional method, such as those previously discussed. Either prior to or after fluid coupling bag assembly 14B to container 220, bag assembly 14B is removed from the centrifuge or the bucket and/or insert thereof. With second platen 436 in the retracted position, bag assembly 14B is then positioned between first platen 434 and second platen 436. Although any of the bag assemblies 14 disclosed or envisioned herein can be used with expressor 430A, bag assembly 14B is shown in FIG. 16. In this assembly, ports 58A1 and 58B1 can be aligned with notches 450A and 450B so that inlet line 90 or outlet line 92 pass therethrough, respectively. Notches 450 function in part to prevent damage to port 58 and kinking of lines 90 and 92 during operation of expressor 430A. If bag assembly 14A is used (FIG. 2), notches 450 are not needed.

Once bag assembly 14B is properly positioned on expressor 430A and fluid coupled with container 220, second platen 436 can be moved toward first platen 434, i.e., toward the collapsed position, so that bag assembly 14B is compressed between platens 434 and 436, thereby driving/forcing the supernatant 216 to flow out of bag assembly 14B, through outlet line 92 and into container 220. More specifically, once bag assembly 14B is properly positioned on expressor 430A and fluid coupled with container 220, handle 466 (FIG. 10) can be released from catch 468 which enables second platen 436 to move toward the collapsed position, i.e., toward the first platen 434, under the force of spring 464 by pivoting about hinge 462. The compressing of bag assembly 14B between platens 434 and 436 under the force of spring 464 drives/forces the supernatant 216 to flow out of bag assembly 14B, through outlet line 92 and into container 220.

In one method of use, the width of gap spacing 480 (FIG. 13) is selectively adjusted prior to use so that as second platen 436 moves to the final collapsed position, pellet 214 is pancaked so as to spread out within bag assembly 14B by being compressed between platens 434 and 436 (FIG. 13). The pancaking and spreading of pellet 214 further drives supernatant 216 out of bag assembly 14B and into container 220. However, gap spacing 480 is typically set so that no portion of pellet 214 flows out of bag assembly 14B and into container 220. That is, gap spacing 480 is set so that when second platen 436 reaches its collapsed position, i.e., second platen 436 is no longer advancing toward first platen 434, the pancaked pellet 214 fills bag assembly 14B up toward ports 58A1 and 58B1 but does not reach or pass out through ports 58A1 and 58B1. As such, pellet 214 cannot flow into outlet line 92. Rather, pancaked pellet 214 only extends up to a level below ports 58A1 and 58B1 so that some supernatant 216 remains within bag assembly 14B and occupies the volume between the top of pancaked pellet 214 and ports 58.

Typically, gap spacing 480 is set so that between 1 ml and 150 ml or between 25 ml and 150 ml and more commonly between 25 ml and 50 ml or between 50 ml and 100 ml of supernatant 216 remains within bag assembly 14 when second platen 436 has reached its final collapsed position. Commonly, the spacing between pancaked pellet 214 and ports 58A1 and 58B1 when second platen 436 is in the collapsed position is less than 4 cm and more commonly less than 3 cm, 2 cm, 1 cm, 0.5 cm, 0.2 cm or less. Other distances can also be used. It is typically preferred to minimize the amount of supernatant 216 that remains within bag 54 so as to optimize separation of supernatant 216 and pellet 214. Adjusting the width of gap spacing 480 is used to help optimize separation of supernatant 216 and pellet 214 by ensuring that pancaked pellet 214 ends close to ports 58A1 and 58B1 when second platen 436 is in the collapsed position. The setting of gap spacing 480 can vary depending on the size/quantity of pellet 214 collected within bag 54. For example, for a fixed sized bag 54, gap spacing 480 can be to be increased as the size/quantity of pellet 214 increases and can be decreased as the size/quantity of pellet 214 decreases.

The amount of pellet 214 within a bag assembly 14 can vary dependent upon a number of different factors, including the volume percentage of cells in suspension 12 withdrawn from the reactor and fed into bag assembly 14. As such, by using spring 464 (FIG. 10) which requires no controller and by selectively adjusting gap spacing 480 dependent upon the quantity of pellet 214 within bag assembly 14B, expressor 430A can freely and independently operate to maximize the transfer supernatant 216 to container 220 with decreased risk of any of pellet 214 flowing into container 220. Thus, expressor 430A provides an inexpensive way to optimize separation pellet 214 and supernatant 216 with minimal monitoring.

Where it is desired to collect and further process and use supernatant 216, it is desirable to prevent any of pellet 214 from flowing into container 220. However, where supernatant 216 is not being used but rather pellet 214 is being collected for further use, it is less critical whether a portion of pellet 214 flows into container 220. For example, it may be desirable to set gap spacing 480 so that a small portion of pellet 214 flows into container 220, thereby helping to ensure that a maximum quantity of supernatant has been removed from bag assembly 14.

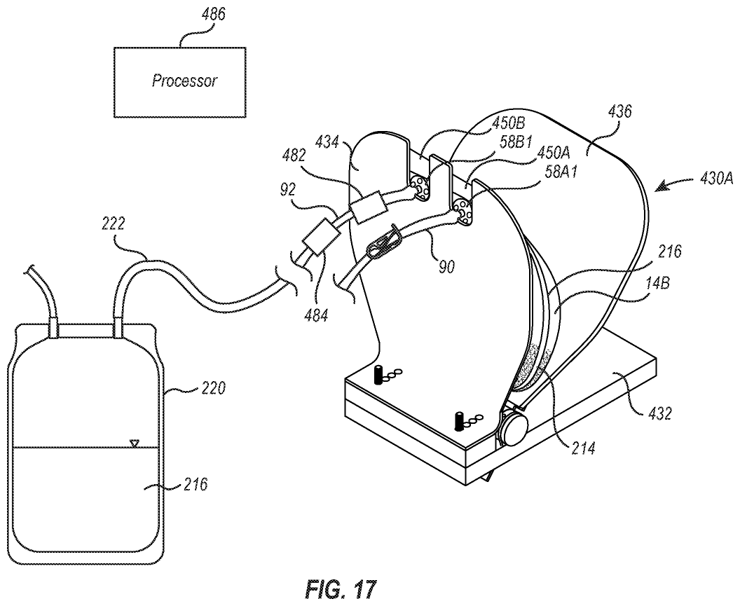

Independent of or in combination with adjusting gap spacing 480 to prevent the unwanted removal of pellet 214 from bag assembly 14, other mechanisms can also be used to prevent the flow of pellet 214 into container 220. For example, as depicted in FIG. 17, outlet line 92 can have an optical sensor 482 overlaying outlet line 92 and an electric pinch clamp 484 overlying outlet line 92 down stream of optical sensor 482. Optical sensor 482 and pinch clamp 484 can be electronically controlled by a processor 486. During operation, while expressor 430A is driving supernatant 216 from bag assembly 14 to container 220, optical sensor 482 in combination with processor 486 monitors the clarity or density of the fluid flowing through outlet line 92.

If processor 486 detects that the fluid flowing through outlet line 92 is starting to become less clear, i.e., more opaque, or has an increase in density, both of which can be signs that a portion of pellet 214 is starting to flow through outlet line 92, processor 486 operates pinch clamp 484 to close outlet line 92, thereby preventing any of pellet 214 from flowing into container 220. Where second platen 436 is begin moved by a motor, as opposed to a resilient spring, processor 486 could also function to simply turn off the motor based on signals from optical sensor 482, thereby again helping to ensure that no portion of pellet 214 reaches container 220. Optical sensor 482 can be replaced with other sensors such as capacitance for conductivity or other sensors that detect the increase in cell content.

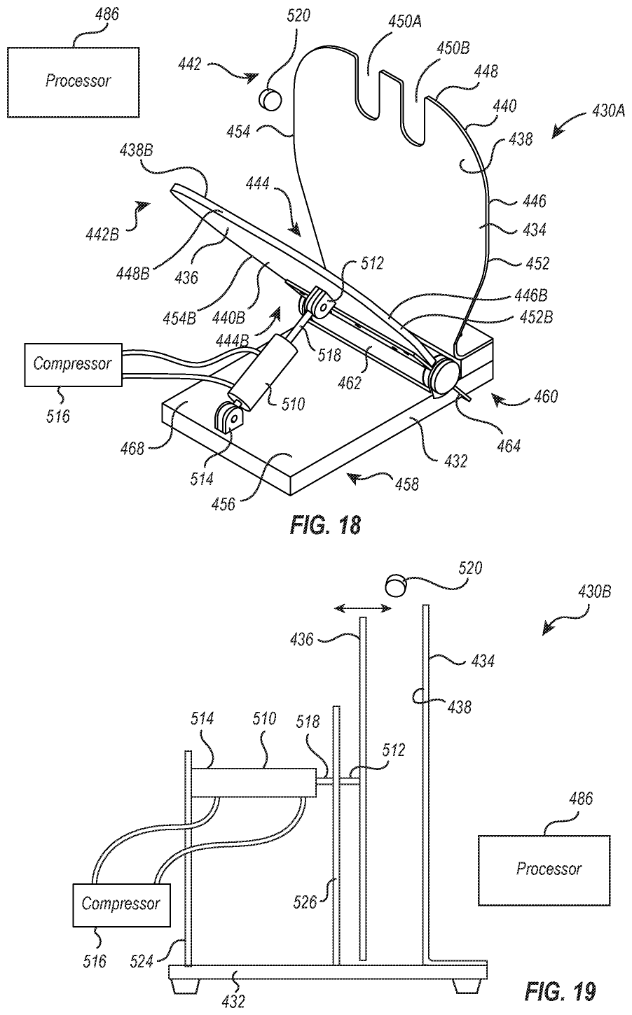

Depicted in FIG. 18 is a modified version of expressor 430A where handle 466 and spring 464 (FIG. 10) have been removed and replaced with a piston 510. Piston 510 can comprise a pneumatic or hydraulic piston and has a first end 512 hingedly coupled to second platen 436 and an opposing second end 514 hingedly coupled to base 432 at first end 458. A compressor 516 is coupled to piston 510 and is used to selectively expand and contract a piston rod 518 of piston 510. The expansion of piston rod 518 moves second platen 436 to the collapsed position and contraction of piston rod 518 moves second platen 436 to the retracted position. Processor 486 can be used to control the movement of piston 510 and thus the movement of second platen 436. Processor 486 can be programmed to move piston 510 back and forth over a fixed distance or can be used with a sensor 520, such as an optical sensor, that senses when second platen 436 has reached the collapsed position. Compressor 516 and piston 510 can also be used with optical sensor 482, pinch clamp 484, and processor 486, as discussed above with regard to FIG. 17. Other methods for controlling the movement of piston 510 can also be used.

Depicted in FIG. 19 is another alternative embodiment of an expressor 430B that includes first platen 434 and second platen 436. Expressor 430B includes base 432 having first platen 434 upstanding therefrom. As discussed above, first platen 434 can be movably mounted on base 432 so as to adjust the gap spacing between platens 434 and 436. Second platen 436 is movably positioned adjacent to first platen 434. However, in contrast to the above embodiments where second platen 436 pivots as it moves between the retracted and collapsed positions, in expressor 430B, second platen 436 moves laterally as it moves between the retracted and collapsed positions. More specifically, inside face 438B of second platen 436 is typically disposed parallel to and remains parallel to inside face inside face 438 of first platen 434 as second platen 436 moves laterally between the retracted and collapsed positions.

In the depicted embodiment, second platen 436 is moved by piston 510 having a piston rod 518. As discussed above, piston 510 can comprise a pneumatic or hydraulic piston and uses a compressor 510 to expand and contract piston rod 518. First end 512 of piston 510 is secured to second platen 436 while second end 514 of piston 510 is secured to a brace 524 upstanding from base 432. A support 526 also upstands from base 432 and supports piston rod 518 that passes therethrough. Processor 486 can be used to control the movement of piston 510 and thus the movement of second platen 436. Processor 486 can be programmed to move piston 510 back and forth over a fixed distance or can be used with sensor 520, such as an optical sensor, that senses when second platen 436 has reached the collapsed position. Compressor 516 and piston 510 can also be used with optical sensor 482, pinch clamp 484, and processor 486, as discussed above with regard to FIG. 17. Other methods for controlling the movement of piston 510 and second platen 436 can also be used.

Expressor 430B works in substantially the same way as expressor 430A.

Specifically, with second platen 436 in the retracted position, bag assembly 14 is positioned between platens 434 and 436. Bag assembly 14 can be supported on one of platens 434 or 436 or can simply be supported on base 432. Second platen 436 is then moved laterally to the collapsed position through the use of compressor 516. As bag 54 is compressed between platens 434 and 436, the supernatant 216 is driven out of bag 54 and into container 220. As discussed above, bag 54 is compressed until the desired amount of supernatant 216 has been removed. In contrast to using piston 510, other types of drive mechanisms, such as a gear assembly, screw drive, worm drive or linkage driven by a motor can be used. In addition, one or more springs or elastic bands can be used to move second platen 436 from the retracted position to the collapsed position.

Where pellet 214 is fragile, further precautionary steps can be taken to prevent disturbing and resuspending portions of pellet 214 as supernatant 216 is removed from bag assembly 14. For example, as shown in FIG. 9, outlet line 92 of bag assembly 14 is again coupled with container 220 through a sterile connection. As previously discussed with regard to FIG. 9, this can be through a direct coupling with container 220 or through inlet line 222 coupled with container 220. This coupling can be accomplished either before or after removing bag assembly 14 from the centrifuge or the buck or insert thereof.

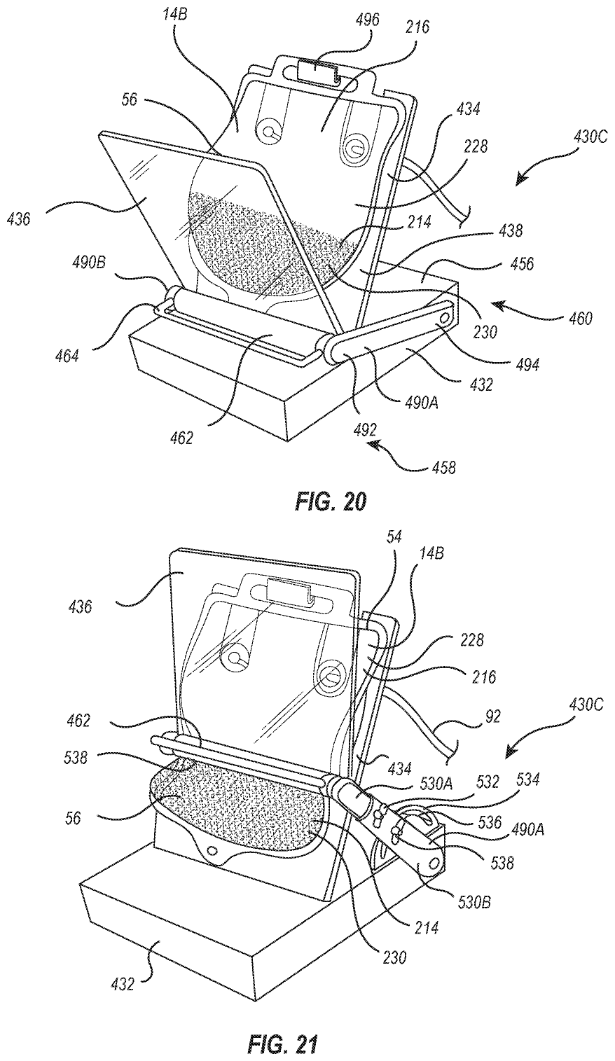

Once bag assembly 14 is fluid coupled within container 220, bag assembly 14 is mounted on an expressor 430C, as shown in FIG. 20. As discussed below in more detail, expressor 430C functions to divide compartment 56 of bag assembly 14B into an upper compartment 228 that houses supernatant 216 and a lower compartment 230 that houses pellet 214. Again, pellet 214 has a higher density than supernatant 216 and can have a higher viscosity. Thus, expressor 430C is applied so that upper compartment 228 holds a first component and lower compartment 230 holds a second component where the second component has a higher density and/or viscosity than the first component. It is appreciated that a small amount of supernatant 216 may be permitted to be retained within lower compartment 230 to minimize disruption of pellet 214 as expressor 430C is attached. Expressor 430C functions to seal upper compartment 228 from lower compartment 230 so that no portion of pellet 214 can pass into upper compartment 228.

Except as noted below, expressors 430A and 430C operate in substantially the same way and like elements between expressors 430A and 430C are identified by like reference characters. Expressor 430C includes base 432 having top surface 456. Upstanding from base 432 is first platen 434. Although first platen 434 could extend orthogonal to base 432, in this embodiment first platen 434 is sloped to form an outside angle between first platen 434 and top surface 456 of base 432 that is greater than 90°. Expressor 430C also includes second platen 436 having a lower end coupled to hinge 462. Spring 464 is coupled to hinge 462 and is used to urge rotation of second platen 436 from the retracted position to the collapsed position. However, in contrast to expressor 430A where hinge 462 is directly secured to base 432, expressor 430C includes a pair of elongated arms 490A and 490B each having a first end 492 and an opposing second end 494. Second ends 494 of arms 490 are rotatably mounted on opposing sides of base 432 at or toward second end 460 of base 432. Hinge 462 extends between first ends 492 of arms 490 so that second platen 436 hingedly rotates relative to arms 490.

During operation, bag assembly 14 is supported against inside face 438 of first platen 434 by being suspended from a hanger 496 extending from first platen 434. Next, arms 490 are rotated upward, as shown in FIG. 21, so that hinge 462 compresses bag assembly 14 against first platen 434 directly above pellet 214, thereby dividing compartment 56 into upper compartment 228 that houses supernatant 216 and a lower compartment 230 that houses pellet 214, as discussed above. Arms 490 are locked in place so as to secure the seal between compartment 228 and compartment 230.

To assist in effectively using hinge 462 to divide compartment 56 into upper compartment 228 and lower compartment 230, arms 490 can be adjustable in length and can lock at the desired length. For example, as shown in FIG. 21, each arm 490 can comprise a first portion 530A and a second portion 530B that slidably couple together, such as by telescoping. Portions 530A and 530B can be locked together in a desired length by a fastener 532. Furthermore, each arm 490 can be rotated to a desired angle and locked in place. For example, as also shown in FIG. 21, a brace 534 can be mounted on base 432 adjacent to arm 490A. An arched slot 536 extends through brace 534. A fastener 538 is slidably received within slot 536 and is connected to arm 490A. For example, fastener 538 can comprise a threaded bolt that passes through slot 536 and through an opening in arm 490A with a nut mounted on the end of the bolt. Arm 490A can be freely rotated relative to base 432 with fastener 538 sliding within slot 536. Once arms 490 are in a desired orientation, fastener 538 can be tightened or otherwise locked in place so as to rigidly secure arm 490A to brace 534, thereby securing arms 490 at the desired angle. Accordingly, by selectively adjusting the length and angle of arms 490, hinge 462 can be securely pressed against bag 54 to divide compartment 56 into compartments 228 and 230. A soft sealing member 538 can also be disposed along the length of hinge 462 to bias against bag 54 for effecting a seal thereat.

Once hinge 462 has been positioned to form compartments 228 and 230, second platen 436 is then permitted to freely rotate under the force of spring 464 so as to compress the portion of bag assembly 14 bounding compartment 228, thereby driving supernatant 216 out of compartment 228, through outlet line 92, and into container 220 (FIG. 9). Expressor 430C thus limits the risk of any portion of pellet 214 flowing into container 220 because pellet 214 is sealed off from supernatant 216.

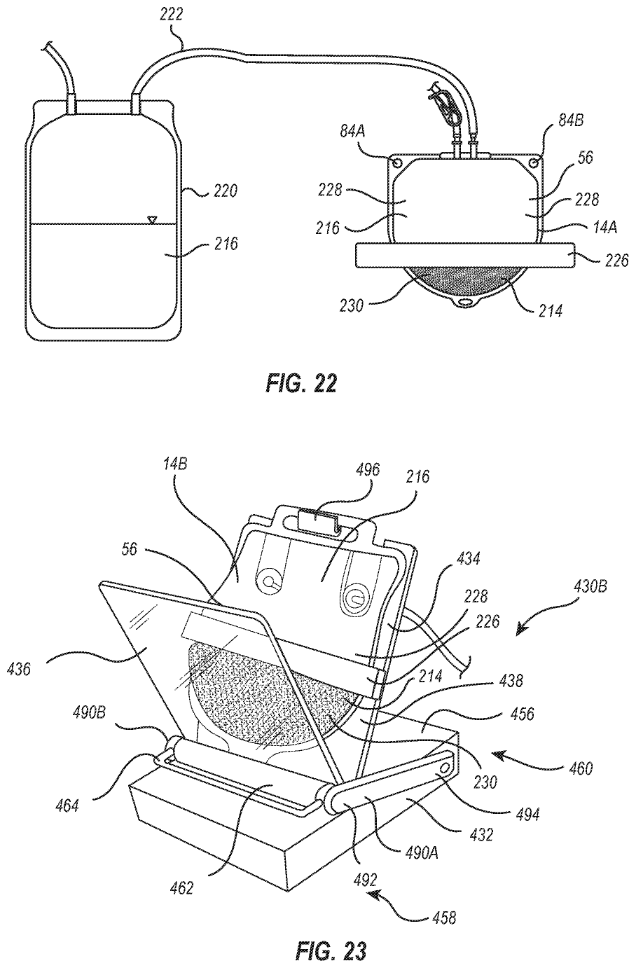

To still further assist in helping to keep supernatant 216 separated from pellet 214, a clamp 226 can be clamped over bag assembly 14 directly above pellet 214 as depicted in FIG. 22. Clamp 226 is typically applied prior to positioning bag assembly 14 on expressor 430C. Clamp 226 functions to divide compartment 56 into upper compartment 228 that houses supernatant 216 and lower compartment 230 that houses pellet 214.

Bag assembly 14B with clamp 226 mounted thereon (FIG. 23) can be suspended on first platen 434 using hanger 496 (FIG. 20). Arms 490 can then be rotated upward, as discussed above, so that hinge 462 extends across bag assembly 14 just above clamp 226. Second platen 436 can then be moved to the collapsed position that compresses upper compartment 228 and drives supernatant 216 into container 220.

In alternative embodiments, other methods can be used to form and seal upper compartment 228 from lower compartment 230. For example, bag assembly 14 could be temporarily pinched closed along the same line that clamp 226 is attached. This can be accomplished by pressing together structural members on opposing sides of bag assembly 14 along the clamp line so as to seal upper compartment 228 from lower compartment 230. In another alternative, bag assembly 14 could be permanently welded closed along the clamp line so to seal upper compartment 228 from lower compartment 230. Again, once upper compartment 228 is isolated from lower compartment 230, supernatant 216 can be dispensed into container 220 without risk of resuspending pellet 214 into supernatant 216. Other methods that seal upper compartment 228 from lower compartment 230 can also be used.

Once the supernatant 216 and pellet 214 separated and isolated, they can be used or discarded as desired. Uses and further processing steps for the separated supernatant 216 and pellet 214 are discussed in U.S. application Ser. No. 16/289,296 (US Patent Publication No.) ______, which was previously incorporated by reference.

The inventive systems disclosed herein have a number unique advantages over the prior art. For example, bag assemblies 14, container 220 and other containers that may be fluid coupled with bag assemblies 14 can all be sterilized prior to use and all fluid couplings formed therewith or therebetween can be sterile connections. The transfer of suspension 12 from reactor 10 into bag assembly 14 and the transfer of the supernatant 216 and pellet 214 out of bag assembly 14 can thus be accomplished without exposing suspension 12 or its components to the open environment or other sources of contaminants. Thus, there is no risk, or at least minimal risk, of suspension 12 or its components being contaminated as they are processed, as set forth above. As a result, there is usually no need for post purification processing of the suspension components (other than, for example, filtering a small amount of residual cells from supernatant 216). The transfer of suspension 12 and separated components through closed lines also reduces the risk that product can be spilled. As such, there is lower risk of losing product by spilling. Delays and efforts in cleaning spilled product is also avoided. This closed processing in a sterile environment is in stark contrast to the prior art where both the original suspension and the formed supernatant are openly exposed to the environment as they are transferred into and out of the bottles or flasks that are used during centrifugation.

Furthermore, biological suspensions have traditionally not been separated by centrifugation within a closed bag to produce a supernatant and pellet. Using an expressor has been found to provide and easy and cost efficient method to remove the supernatant from the bag without removing the pellet while retaining the pellet and/or the supernatant sterile.

Although some of the expressors disclosed herein after have some components similar conventional plasma expressors, the disclosed expressors have unique features. For example, bag assemblies are 14B are uniquely configured having ports 58A1 and 58B1 formed on the front face thereof (FIG. 4). In part, as previously discussed herein, ports 58A1 and 58B1 are so positioned so as to help prevent damage or leakage during centrifugal rotation of bag assembly 14B. In turn, notches 450 can be formed on first or section platens so receive ports 58A1 and 58B1, thereby preventing blocking or kinking of the ports or lines extending therefrom during compression and better enabling uniform compression of bag assembly 14.

In addition, bag assemblies 14 are tapered to help optimize the formation of a consolidated pellet within bag assemblies 14. In turn, the platens of the disclosed expressors can be formed with a complementary taper to again help enable uniform compression of the bag assemblies while minimizing cost and limiting any obstructions to visualization of the bag assemblies.

Furthermore, blood bags used for separating plasma house blood which has a substantially constant concentration of plasma. As such, there is no need in plasma expressors to be able to adjust the gap spacing between adjacent platens. In contrast, the bag assemblies of the present disclosure can have wide fluctuations in the percent volumes of supernatant and pellet that are produced therein. Having the ability to adjust the spacing between platens based on the quantity of pellet within the bag assembly improves optimization of separating the supernatant from the pellet while minimizing risk that a portion of the pellet will outflow with the supernatant.

Furthermore, the use of expressor 430C with or without clamp 226 or the other sealing mechanisms discussed herein provides an easy mechanism for isolating the supernatant from the pellet so that the pellet does not resuspend into the supernatant as the supernatant is removed from the bag assembly. This is particularly useful where the pellet is loose and easily resuspended. Accordingly, using expressor 430C and the other sealing mechanisms both increases the quality of the supernatant that can be removed and shortens production time.

Various alterations and/or modifications of the inventive features illustrated herein, and additional applications of the principles illustrated herein, which would occur to one skilled in the relevant art and having possession of this disclosure, can be made to the illustrated embodiments without departing from the spirit and scope of the invention as defined by the claims, and are to be considered within the scope of this disclosure. Thus, while various aspects and embodiments have been disclosed herein, other aspects and embodiments are contemplated. While a number of methods and components similar or equivalent to those described herein can be used to practice embodiments of the present disclosure, only certain components and methods are described herein.

It will also be appreciated that systems, processes, and/or products according to certain embodiments of the present disclosure may include, incorporate, or otherwise comprise properties features (e.g., components, members, elements, parts, and/or portions) described in other embodiments disclosed and/or described herein. Accordingly, the various features of certain embodiments can be compatible with, combined with, included in, and/or incorporated into other embodiments of the present disclosure. Thus, disclosure of certain features relative to a specific embodiment of the present disclosure should not be construed as limiting application or inclusion of said features to the specific embodiment. Rather, it will be appreciated that other embodiments can also include said features without necessarily departing from the scope of the present disclosure.

Moreover, unless a feature is described as requiring another feature in combination therewith, any feature herein may be combined with any other feature of a same or different embodiment disclosed herein. Furthermore, various well-known aspects of illustrative systems, processes, products, and the like are not described herein in particular detail in order to avoid obscuring aspects of the example embodiments. Such aspects are, however, also contemplated herein.

The present disclosure may be embodied in other specific forms without departing from its spirit or essential characteristics. The described embodiments are to be considered in all respects only as illustrative and not restrictive. The scope of the invention is, therefore, indicated by the appended claims rather than by the foregoing description. While certain embodiments and details have been included herein and in the attached disclosure for purposes of illustrating embodiments of the present disclosure, it will be apparent to those skilled in the art that various changes in the methods, products, devices, and apparatus disclosed herein may be made without departing from the scope of the disclosure or of the invention, which is defined in the appended claims. All changes which come within the meaning and range of equivalency of the claims are to be embraced within their scope.

What is claimed is:

1. An expressor comprising:
a base;
a first platen having an inside face extending between an upper end and an opposing lower end, the lower end extending from the base;
a second platen having an inside face extending between an upper end and an opposing lower end, the second platen being movably mounted to the base so that the second platen can move between a collapsed position where the second platen is moved toward the first platen and a retracted position where the second platen is moved away from the first platen, at least a portion of the second platen being spaced apart from the first platen by a gap spacing when the second platen is in the collapsed position,
wherein either the first platen or the second platen is releasably attached to the base so that a width of the gap spacing when the second platen is in the collapsed position can be selectively adjusted.

2. The expressor as recited in claim 1, wherein the lower end of the second platen is pivotably connected to the base so that the second platen can pivot between the collapsed position and the retracted position.

3. The expressor as recited in claim 1, wherein either the first platen or the second platen is releasably attached to the base so that the width of the gap spacing can be selectively adjusted without pivoting of the first platen or the second platen.

4. The expressor as recited in claim 1, wherein the inside face of the first platen is disposed in parallel alignment with the inside face of the second platen when the second platen is in the collapsed position.

5. The expressor as recited in claim 1, further comprising means for moving the second platen toward the first platen.

6. The expressor as recited in claim 5, wherein the means for moving comprises a spring, pneumatic piston or hydraulic piston.

7. The expressor as recited in claim 1, wherein the first platen inwardly tapers at the lower end.

8. An expressor system comprising:
the expressor as recited in claim 1; and
a bag assembly comprising:
a collapsible bag bounding a compartment that is adapted to hold a fluid, the bag being disposed between the first platen and the second platen; and
a tube projecting from the collapsible bag.

9. The expressor system as recited in claim 8, further comprising a pellet comprised of cells or microorganisms disposed within the compartment of the bag and a liquid supernatant disposed within the compartment of the bag, wherein the pellet comprises cells that are free red blood cells and white blood cells and the liquid supernatant is free of plasma.

10. A method using the expressor recited in claim 1 for removing a supernatent from a compartment of a collapsible bag that houses the supernatant and a pellet comprised of cells or microorganisms, the method comprising:
moving the first platen or the second platen of the expressor recited in claim 1 relative to the base so as to adjust the width of the gap spacing between the first platen and the second platen based upon an amount of pellet within the compartment of the bag;
positioning the collapsible bag between the first platen and the second platen; and
moving the second platen toward the first platen so as to compress the bag between the first platen and the second platen and drive at least a portion of the supernatant out of the bag through a tube coupled with the bag.

11. An expressor comprising:
a base;
a first platen having an inside face and an opposing outside face extending between an upper end and an opposing lower end, the upper end having a perimeter edge with a first notch recessed into the perimeter edge so that the first notch passes between the inside face and the outside face, the lower end being connected to the base;
a second platen having an inside face extending between an upper end and an opposing lower end, the lower end of the second platen being movably mounted to the base so that the second platen can move between a collapsed position where the second platen is moved toward the first platen and a retracted position where the second platen is moved away from the first platen.

12. The expressor as recited in claim 11, wherein the second platen is pivotably connected to the base so that the second platen can pivot between the collapsed position and the retracted position.

13. The expressor as recited in claim 11, further comprising a second notch recessed into the perimeter edge at the upper end of the first platen so that the second notch passes between the inside face and the outside face, the second notch being spaced apart from the first notch.

14. The expressor as recited in claim 11, further comprising means for moving the second platen between the collapsed position and the retracted position.

15. An expressor system comprising:
the expressor as recited in claim 11; and
a bag assembly comprising:
a collapsible bag bounding a compartment that is adapted to hold a fluid, the bag having a front face and an opposing back face, the bag being disposed between the first platen and the second platen;
a first port being disposed on the front face of the bag and communicating with the compartment, the first port being received within the first notch of the first platen;
a tube projecting from the first port.

16. The expressor system as recited in claim 15, further comprising a pellet comprised of cells or microorganisms disposed within the compartment of the bag and a liquid supernatant disposed within the compartment of the bag.

17. A method using the expressor recited in claim 11 for removing a supernatant from a compartment of a collapsible bag that houses the supernatant and a pellet comprised of cells or microorganisms, the method comprising:
positioning the collapsible bag between the first platen and the second platen of the expressor recited in claim 11, a first port being disposed on the front face of the bag and communicating with the compartment, the first port being received within the first notch of the first platen; and
moving the second platen toward the first platen so as to compress the bag between the first platen and the second platen and drive at least a portion of the supernatant out of the bag through a tube coupled with the bag.

18. An expressor comprising:
a base;
a first platen having an inside face extending between an upper end and an opposing lower end, the lower end extending from the base;
a first arm and a spaced apart second arm movably mounted to the base;
a second platen having an inside face extending between an upper end and an opposing lower end, the second platen being pivotably connected to the first arm and the second arm so that the second platen can pivot toward and away from the first platen.

19. The expressor as recited in claim 18, wherein the first arm and the second arm each have a first end and an opposing second end, the first end of each arm being pivotably connected to the base and the second end of each arm being pivotably connected to the second platen.

20. An expressor system comprising:
the expressor as recited in claim 18; and
a bag assembly comprising a collapsible bag bounding a compartment that is adapted to hold a liquid, the bag being disposed between the first platen and the second platen, the second platen being elevated by the first arm and the second arm so as to be spaced apart from the base.
